(19) United States
(12) Patent Application Publication
(10) Pub. No.: US 2022/0370527 A1
(43) Pub. Date: Nov. 24, 2022

(54) DELIVERY OF SIALIDASE TO CANCER CELLS, IMMUNE CELLS AND THE TUMOR MICROENVIRONMENT

(71) Applicant: Ansun Biopharma, Inc., San Diego, CA (US)

(72) Inventor: Nancy Chang, San Diego, CA (US)

(21) Appl. No.: 17/260,812

(22) PCT Filed: Jul. 22, 2019
(86) PCT No.: PCT/US2019/042848

§ 371 Date: Jan. 15, 2021

Related U.S. Application Data
(60) Provisional application No. 62/796,518, filed on Jan. 24, 2019.
(60) Provisional application No. 62/701,481, filed on Jul. 20, 2018.

(51) Int. Cl. A61K 35/768; C12N 7/00; C12N 9/24; A61P 35/00

(52) U.S. Cl. CPC A61K 35/768; C12N 7/00; C12N 9/2402; A61P 35/00; C12N 2710/24132; C12Y 302/01018; C12Y 302/01129

(57) ABSTRACT

Recombinant oncolytic viruses for expression of sialidase and their use in the treatment of cancer, particularly solid tumors, are described.

### CLAIM OF PRIORITY

This application is a U.S. National Phase application under 35 U.S.C. § 371 of International Patent Application No. PCT/US2019/042848, filed Jul. 22, 2019, which claims the benefit of U.S. Provisional Patent Application Ser. No. 62/796,518, filed Jan. 24, 2019 and Ser. No. 62/701,481, filed Jul. 20, 2018. The entire contents of each of the foregoing applications are hereby incorporated by reference.

### BACKGROUND

Cancer is the second leading cause of death in the United States. In recent years, great progress has been made in cancer immunotherapy, including immune checkpoint inhibitors, T cells with chimeric antigen receptors, and oncolytic viruses.

Oncolytic viruses are naturally occurring or genetically modified viruses that infect, replicate in, and eventually kill cancer cells while leaving healthy cells unharmed. A recently completed Phase III clinical trial of the oncolytic herpes simplex virus T-VEC in 436 patients with unresectable stage IIIB, IIIC or IV melanoma was reported to meet its primary end point, with a durable response rate of 16.3% in patients receiving T-VEC compared to 2.1% in patients receiving GM-CSF. Based on the results from this trial, FDA approved T-VEC in 2015.

Oncolytic virus constructs from at least eight different species have been tested in various phases of clinical trials, including adenovirus, herpes simplex virus-1, Newcastle disease virus, reovirus, measles virus, coxsackievirus, Seneca Valley virus, and vaccinia virus. It has become clear that oncolytic viruses are well tolerated in patients with cancer. The clinical benefits of oncolytic viruses as stand-alone treatments, however, remain limited. Due to concerns on the safety of oncolytic viruses, only highly attenuated oncolytic viruses (either naturally avirulent or attenuated through genetic engineering) have been used in both preclinical and clinical studies. Since the safety of oncolytic viruses has now been well established it is time to design and test oncolytic viruses with maximal anti-tumor potency. Oncolytic viruses with a robust oncolytic effect will release abundant tumor antigens, resulting in a strong immunotherapeutic effect.

### SUMMARY

Provided herein are compositions comprising a recombinant oncolytic virus comprising a nucleic acid molecule encoding one or more human or bacterial sialidases or a functional portion thereof. The oncolytic viruses can be derived from a poxvirus, an adenovirus, a herpes virus or any other suitable oncolytic virus. Suitable recombinant oncolytic viruses can be created by inserting an expression cassette that includes a sequence encoding a sialidase or a portion thereof with sialidase activity into an oncolytic virus.

Many cancer cells are hypersialylated. The recombinant oncolytic viruses described herein are capable of delivering sialidase to tumor cells and the tumor cell environment. The delivered sialidase can reduce sialic acid present on tumor cells and render the tumor cells more vulnerable to killing by immune cells, immune cell-based therapies and other therapeutic agents whose effectiveness is diminished by hypersialylation of cancer cells.

Also provided are methods for delivering a sialidase to the tumor microenvironment. Within the tumor microenvironment the sialidase can remove terminal sialic acid residues on cancer cells, thereby reducing the barrier for entry of immunotherapy reagents and promote cellular immunity against cancer cells.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus, poxvirus), an envelope including lipids and optionally components of host cell membranes, and/or viral proteins.

The term “poxvirus” is used according to its plain ordinary meaning within Virology and refers to a member of Poxviridae family capable of infecting vertebrates and invertebrates which replicate in the cytoplasm of their host. In embodiments, poxvirus virions have a size of about 200 nm in diameter and about 300 nm in length and possess a genome in a single, linear, double-stranded segment of DNA, typically 130-375 kilobase.

The term poxvirus includes, without limitation, all genera of poxviridae (e.g., betaentomopoxvirus, yatapoxvirus, cervidpoxvirus, gammaentomopoxvirus, leporipoxvirus, suipoxvirus, molluscipoxvirus, crocodylidpoxvirus, alphaentomopoxvirus, capripoxvirus, orthopoxvirus, avipoxvirus, and parapoxvirus). In embodiments, the poxvirus is an orthopoxvirus (e.g., smallpox virus, vaccinia virus, cowpox virus, monkeypox virus), parapoxvirus (e.g., orf virus, pseudocowpox virus, bovine popular stomatitis virus), yatapoxvirus (e.g., tanapox virus, yaba monkey tumor virus) or molluscipoxvirus (e.g., molluscum contagiosum virus). In embodiments, the poxvirus is an orthopoxvirus (e.g., cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, or vaccinia virus strain AS). In embodiments, the poxvirus is a parapoxvirus (e.g., orf virus strain NZ2 or pseudocowpox virus strain TJS).

A “sialidase catalytic domain protein” is a protein that comprises the catalytic domain of a sialidase, or an amino acid sequence that is substantially homologous to the catalytic domain of a sialidase, but does not comprise the entire amino acid sequence of the sialidase the catalytic domain is derived from, wherein the sialidase catalytic domain protein retains substantially the same activity as the intact sialidase the catalytic domain is derived from. A sialidase catalytic domain protein can comprise amino acid sequences that are not derived from a sialidase, but this is not required. A sialidase catalytic domain protein can comprise amino acid sequences that are derived from or substantially homologous to amino acid sequences of one or more other known proteins, or can comprise one or more amino acids that are not derived from or substantially homologous to amino acid sequences of other known proteins.

### DESCRIPTION OF THE DRAWINGS

FIG. 1: Detection of 2,6 sialic acid (by FITC-SNA) on A549 and MCF cells by fluorescence microscopy. A549 and MCF cells were fixed and incubated with FITC-SNA for one hour at 37° C. before imaged under fluorescence microscope to show the FITC-SNA labeled cells (left) and overlay with brightfield cells (right)

FIG. 2: Effective removal of 2,6 sialic acid, 2,3 sialic acid, and exposure of galactose on A549 cells by DAS181 treatment. A549 were treated with DAS181 for two hours at 37° C. and incubated with staining reagents one hour before imaged under fluorescence microscope to show effective removal of sialic acids on tumor cells.

FIG. 3: Effective removal of 2,6 sialic acid on A549 cells by DAS181 but not DAS185 treatment. A549 were treated with DAS181 for 30 minutes or two hours at 37° C. and incubated with FITC-SNA for one hour before examined using flow cytometry to show effective removal of 2,6 sialic acids on tumor cells.

FIG. 4: Effective removal of 2,3 sialic acid on A549 cells by DAS181 but not DAS185 treatment. A549 were treated with DAS181 for 30 minutes or two hours at 37° C. and incubated with FITC-MALII for one hour before examined using flow cytometry to show effective removal of 2,3 sialic acids on tumor cells

FIG. 5: Effective exposure of galactose on A549 cells by DAS181 but not DAS185 treatment. A549 were treated with DAS181 for 30 minutes or two hours at 37° C. and incubated with FITC-PNA for one hour before examined using flow cytometry to show effective exposure of galactose on tumor cells

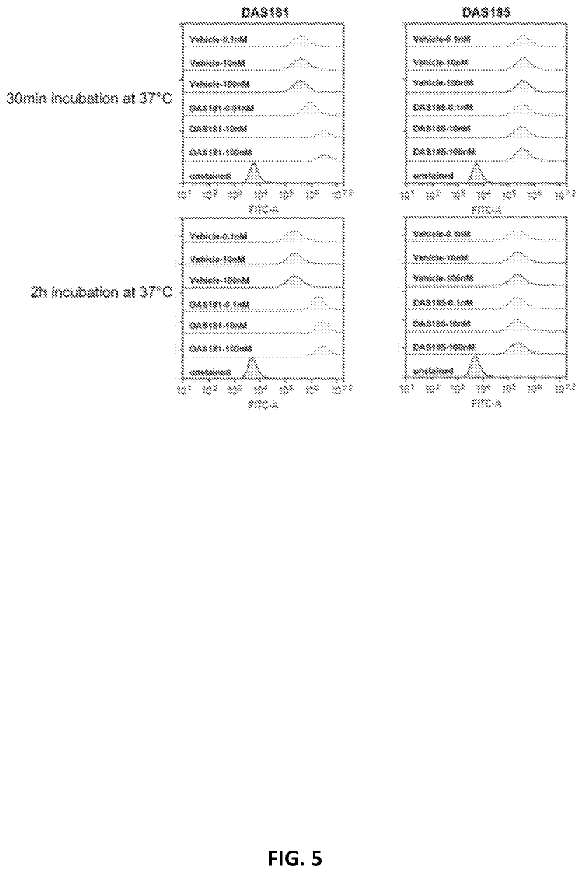

FIG. 6: DAS181 treatment and PBMC stimulation regimen do not affect A549-red cell proliferation. A549-Red cells were seeded at 2 k/well overnight, followed by replacement of medium containing reagents listed on the left. Scan by IncuCyte was initiated immediately after the reagents were added (0 hr) and scheduled for every 3 hr. A549-red cell proliferation is monitored by analyzing the nuclear (red) counts. Kinetic readouts reveal no effect on A549 cell proliferation by vehicle, DAS181, or various stimulation reagents, without the presence of PBMCs.

FIG. 7: Detection of cytotoxicity in A549-red cells following co-culturing with PBMCs from Donor 1 with or without DAS181 treatment. These results showed that DAS181 treatment significantly boost anti-tumor cytotoxicity by PBMCs from Donor 1. A549-Red cells were seeded at 2 k/well overnight, followed by co-culturing with 100K/well Donor-1 PBMCs (E:T=50:1) in the presence of medium (no activation), CD3+CD28+IL-2 (T cell activation), or CD3+CD29+IL-2+IL-15+IL-21 (T and NK cell activation). Representative images were taken by IncuCyte at 0 hr and 72 hrs post adding PBMCs.

FIG. 8: Detection of cytotoxicity in A549-red cells following co-culturing with PBMCs from Donor 2 with or without DAS181 treatment. These results showed that DAS181 treatment significantly boost anti-tumor cytotoxicity by PBMCs from Donor 2. A549-Red cells were seeded at 2 k/well overnight, followed by co-culturing with 100 k/well Donor-1 PBMCs (E:T=50:1) in the presence of medium (no activation), CD3+CD28+IL-2 (T cell activation), or CD3+CD29+IL-2+IL-15+IL-21 (T and NK cell activation). Representative images were taken by IncuCyte at 0 hr and 72 hrs post adding PBMCs.

FIGS. 9A-9C: Detection of cytotoxicity in A549-red cells following co-culturing with PBMCs from Donor 1 with or without DAS181 treatment. These results showed that DAS181 treatment significantly boost anti-tumor cytotoxicity by PBMCs from Donor 1. The green lines indicate conditions without DAS181, and the blue lines indicate conditions with the DAS-181 treatment. A549-red tumor cells were seeded at 2 k cells/well in 96-well plate. After overnight incubation, PBMCs from Donor 1 mixed with (A) medium (B) CD3/CD28/IL-2, or (C) CD3/CD28/IL-2/IL-15/IL-21 were added into each well as indicated E:T ratio. At mean time, DAS181 (100 nM) was added. Plates were scanned by IncuCyte every 3 hr for total 72 hrs. Proliferation is monitored by analyzing RFP cell counts.

FIGS. 10A-10C: Detection of cytotoxicity in A549-red cells following co-culturing with PBMCs from Donor 2 with or without DAS181 treatment. These results showed that DAS181 treatment significantly boost anti-tumor cytotoxicity by PBMCs from Donor 2. The green lines indicate conditions without DAS181, and the blue lines indicate conditions with the DAS-181 treatment. A549-red tumor cells were seeded at 2 k cells/well in 96-well plate. After overnight incubation, PBMCs from Donor 2 mixed with (A) medium, (B) CD3/CD28/IL-2, or (C) CD3/CD28/IL-2/IL-15/IL-21 were added into each well as indicated E:T ratio. At mean time, DAS 181 (100 nM) was added. Plates were scanned by IncuCyte every 3 hr for total 72 hrs. Proliferation is monitored by analyzing RFP cell counts.

FIG. 11: DAS181 enhances NK-mediated tumor lysis by vaccinia virus, measured by MTS assay. [symbol] =T-test P value<0.05, suggesting that DAS181 alone boosts NK cell-mediated U87 tumor killing in vitro, compared to enzyme-dead DAS185. *=T-Test P value<0.05.

FIG. 12: DAS181 increases NK-mediated tumor killing by vaccinia virus as measured by MTS assay. *=T-test P value<0.05, suggesting that DAS181 increases NK cell-mediated killin of U87 cells by VV in vitro.

FIG. 13: DAS181 significantly enhanced expression of maturation markers (CD80, CD86, HLA) in human DC cells that were cultured alone or exposed to VV-infected tumor cells. *=T-test P value<0.05.

FIG. 14: DAS181 significantly enhanced TNF-alpha production by THP-1 derived macrophages. *=T-test P value<0.05

FIG. 15: DAS181 treatment promotes oncolytic adenovirus-mediated tumor cell killing and growth prohibition. A549-red tumor cells were seeded at 2K cells/well in 96-well plates. After overnight incubation, DAS181 vehicle, oncolytic adenovirus, and DAS181 were added as indicated. CD3/CD28/IL-2 were also added into each well with the amount described previously. Graph showed that DAS181 plus oncolytic adenovirus effectively reduced tumor cell proliferation.

FIGS. 16A-16B: DAS181 treatment enhances PBMC-mediated tumor cell killing by vaccinia virus. A549-red tumor cells were seeded at 2K cells/well in 96-well plate. After overnight incubation, fresh PBMCs were added at densities of 10K/well (A) or 40K/well (B). CD3, CD28, IL-2, DAS181, and oncolytic adenovirus were added as indicated in the graph following with the timed scans by IncuCyte. Graph showed that DAS181 plus oncolytic adenovirus dramatically enhanced human PBMC-mediated tumor cell eradication.

FIG. 17: Schematic of a portion of a vaccinia virus construct for expressing a sialidase.

FIG. 18: Sequence of certain elements in a vaccinia virus construct for expressing a sialidase (DAS181).

FIG. 19: Sequence of a portion of a vaccinia virus construct for expressing a sialidase (DAS181).

FIGS. 20A-20B: DAS181 expressed by Sialidase-VV has in vitro activity towards sialic acid-containing substrates. (A) Standard curve of DAS181 activity at 0.5 nM, 1 nM, and 2 nM. (B) 1×10⁶ cells infected with Sialidase-VV express DAS181 equivalent to 0.78 nM-1.21 nM DAS181 in 1 ml medium in vitro.

FIG. 21: Sialidase-VV enhances Dendritic cell maturation. GM-CSF/IL4 derived human DC were cultured with Sial-VV or VV infected U87 tumor cell lysate for 24 hours. DAS181 of LPS was used as control DC were collected and stained with antibodies against CD80, CD86, HLA-DR, and HLA-ABC. The expression of DC maturation markers was determined by flow analysis. The results suggested that Sial-VV enhanced DC maturation. *=T-test P value<0.05

FIG. 22: Sialidase-VV induced IFN-gamma and IL2 expression by T cells. CD3 antibody-activated human T cells were co-cultured with A594 tumor cells in the presence of Sial-VV or VV-infected tumor cells lysate for 24 hours, and cytokine IFNr or IL-2 expression was measured by ELISA. The results suggested that Sial-VV cell lysate induced IFNr and IL2 expression by human T cells. *=T-test P value<0.05

FIG. 23: Sialidase-VV enhances T cell-mediated tumor cell lytic activity. CD3 Ab activated human T cells were co-cultured with Sial-VV or VV-infected A594 tumor cells for 24 hours, and tumor cell viability was determined by MTS assay. The results suggested that Sial-VV infection of tumor cells resulted in enhanced tumor killing. *=T-test P value<0.05

### DETAILED DESCRIPTION

### Oncolytic Viruses

Numerous oncolytic viruses, including Vaccina virus, Coxsackie virus, Adenovirus, Measles, Newcastle disease virus, Seneca Valley virus, Coxsackie A21, Vesicular stomatitis virus, Parvovirus H1, Reovirus, Herpes virus, Lentivirus, and Poliovirus, and Parvovirus. Vaccinia Virus Western Reserve, GLV-1h68, ACAM2000, and OncoVEX GFP, are available. The genomes of these oncolytic virus can be genetically modified to insert a nucleotide sequence encoding a protein that includes all or a catalytic portion of a sialidase. The nucleotide sequence encoding a protein that includes all or a catalytically active portion of a sialidase is placed under the control of a viral expression cassette so that the sialidase is expressed by infected cells.

### Vesicular Stomatitis Virus (VSV)

VSV has been used in multiple oncolytic virus applications. In addition, VSV has been engineered to express an antigenic protein of human papilloma virus (HPV) as a method to treat HPV positive cervical cancers via vaccination (REF 18337377, 29998190) and to express pro-inflammatory factors to increase the immune reaction to tumors (REF 12885903). Various methods for engineering VSV to encode an additional gene have been described (REF 7753828). Briefly, the VSV RNA genome is reverse transcribed to a complementary, doubled stranded-DNA with an upstream T7 RNA polymerase promoter and an appropriate location within the VSV genome for gene insertion is identified (e.g., within the noncoding 5′ or 3′ regions flanking VSV glycoprotein (G) (REF 12885903). Restriction enzyme digestion can be accomplished, e.g., with Mlu I and Nhe I, yielding a linearized DNA molecule. An insert consisting of a DNA molecule encoding the gene of interest flanked by appropriate restriction sites can be ligated into the linearized VSV genomic DNA. The resulting DNA can be transcribed with T7 polymerase, yielding a complete VSV genomic RNA containing the inserted gene of interest. Introduction of this RNA molecule to a mammalian cell, e.g., via transfection and incubation results in viral progeny expressing the protein encoded by the gene of interest.

### Adenovirus Serotype 5 (Ad5)

Ad5 contains a human E2F-1 promoter, which is a retinoblastoma (Rb) pathway—defective tumor specific transcription regulatory element that drives expression of the essential Ela viral genes, restricting viral replication and cytotoxicity to Rb pathway-defective tumor cells (REF 16397056). A hallmark of tumor cells is Rb pathway defects. Engineering a gene of interest into Ad5 is accomplished through ligation into Ad5 genome. A plasmid containing the gene of interest is generated via and digested, e.g., with AsiSI and PacI. An Ad5 DNA plasmid, e.g., PSF-AD5 (REF Sigma OGS268) is digested with AsiSI and PacI and ligated with recombinant bacterial ligase or co-transformed with RE digested gene of interest into permissive E. coli as has been reported for the generation of human granulocyte macrophage colony stimulating factor (GM-CSF) expressing Ad5 (REF 16397056). Recovery of the DNA and transfection into a permissive host, e.g., human embryonic kidney cells (HEK293) or HeLa yields virus expressing the gene of interest.

### Vaccinia Virus (VV)

Various strains of VV have been used as templates for OV therapeutics; the unifying feature is deletion of the viral thymidine kinase (TK) gene, rendering a virus dependent upon actively replicating cells, i.e. neoplastic cells, for productive replication and thus these VVs have preferential infectivity of cancer cells exemplified by the Western Reserve (WR) strain of VV (REF 25876464). Production of VV's with a gene of interest inserted in the genome is accomplished with homologous recombination utilizing lox sites, as described in greater detail below.

Polypeptides with Sialidase Activity for Expression by an Oncolytic Virus

The recombinant oncolytic virus expresses a polypeptide that includes all or a catalytic portion of a sialidase that is capable of removing sialic acid (N-acetylneuraminic acid (Neu5Ac)) from a glycan on a human cell. In general, Neu5Ac is linked via an alpha 2,3, an alpha 2,6 or alpha 2,8 linkage to the penultimate sugar in glycan on a protein by any of a variety of sialyl transferases. The common human sialyltransferases are summarized in Table 1.

TABLE I
Nomenclature of Neu5Ac sialyltransferases

| | | | EC | |
|---|---|---|---|---|
| Abbreviation | Resulting Group | Substrate | Number | HGNC |
| ST3Gal I | Neu5Ac-α-(2,3) Gal | Gal-β-1,3-GalNAc | 2.4.99.4 | 10862 |
| ST3Gal II | Neu5Ac-α-(2,3) Gal | Gal-β-1,3-GalNAc | 2.4.99.4 | 10863 |
| ST3Gal III | Neu5Ac-α-(2,3) Gal | Gal-β-1,3 | 2.4.99.6 | 10866 |
| | | (4)-GlcNAc | | |
| ST3Gal IV | Neu5Ac-α-(2,3) Gal | Gal-β-1,4-GlcNAc | 2.4.99.9 | 10864 |
| ST3Gal V | Neu5Ac-α-(2,3) Gal | Gal-β-1,4-Glc | 2.4.99.9 | 10872 |
| ST3Gal VI | Neu5Ac-α-(2,3) Gal | Gal-β-1,4-GlcNAc | 2.4.99.9 | 18080 |
| ST6Gal I | Neu5Ac-α-(2,6) Gal | Gal-β-1,4-GlcNAc | 2.4.99.1 | 10860 |
| ST6Gal II | Neu5Ac-α-(2,6) Gal | Gal-β-1,4-GlcNAc | 2.4.99.2 | 10861 |
| ST6GalNAc | Neu5Ac-α-(2,6) | GalNAc-α-1, | 2.4.99.7 | 23614 |
| I | GalNAc | O-Ser/Thr | | |
| ST6GalNAc | Neu5Ac-α-(2,6) c | Gal-β-1,3-GalNAc- | 2.4.99.7 | 10867 |
| II | GalNA | α-1,O-Ser/Tbr | | |
| ST6GalNAc | Neu5Ac-α-(2,6) | Neu5Ac-α-2,3-Gal- | 2.4.99.7 | 19343 |
| III | GalNAc | β-1,3-GalNAc | | |
| ST6GalNAc | Neu5Ac-α-(2,6) | Neu5Ac-α-2,3Gal- | 2.4.99.7 | 17846 |
| IV | GalNAc | β-1,3-GalNAc | | |
| ST6GalNAc | Neu5Ac-α-(2,6) | Neu5Ac-α-2, | 2.4.99.7 | 19342 |
| V | GalNAc | 6-GalNAc- | | |
| | | β-1,3-GalNAc | | |
| ST6GalNAc | Neu5Ac-α-(2,6) | All α-series | 2.4.99.7 | 23364 |
| VI | GalNAc | gangliosides | | |
| ST8Sia I | Neu5Ac-α-(2,8)- | Neu5Ac-α-2, | 2.4.99.8 | 10869 |
| | Neu5Ac | 3-Gal-β-1, | | |
| | | 4-Glc-β-1, | | |
| | | 1Cer (GM3) | | |
| ST8Sia II | Neu5Ac-α-(2,8)- | Neu5Ac-α- | 2.4.99.8 | 10870 |
| | Neu5Ac | 2,3-Gal-β-1, | | |
| | | 4-GlcNAc | | |
| ST8Sia III | Neu5Ac-α-(2,8)- | Neu5Ac-α-2, | 2.4.99.8 | 14269 |
| | Neu5Ac | 3-Gal-β-1, | | |
| | | 4-GlcNAc | | |
| ST8Sia IV | Neu5Ac-α-(2,8)- | (Neu5Ac-α-2,8) | 2.4.99.8 | 10871 |
| | Neu5Ac | nNeu5Ac- | | |
| | | α-2,3-Gal-β-1-R | | |
| ST8Sia V | Neu5Ac-α-(2,8)- | GM1b, GT1b, | 2.4.99.8 | 17827 |
| | Neu5Ac | GD1a, GD3 | | |
| ST8Sia VI | Neu5Ac-α-(2,8)- | Neu5Ac-α- | 2.4.99.8 | 23317 |
| | Neu5Ac | 2,3(6)-Gal | | |

HGNC: Hugo Gene Community Nomenclature (www.genenames.org)

Domains within Polypeptides Having Sialidase Activity

The expressed polypeptide, in addition to the sialidase or catalytic portion thereof can, optionally, include peptide or protein sequences that contribute to the therapeutic activity of the protein. For example, the protein can include an anchoring domain that promotes interaction between the protein and a cell surface. The anchoring domain and sialidase domain can be arranged in any appropriate way that allows the protein to bind at or near a target cell membrane such that the therapeutic sialidase can exhibit an extracellular activity that removes sialic acid residues. The protein can have more than one anchoring domains. In cases in which the polypeptide has more than one anchoring domain, the anchoring domains can be the same or different. The protein can have more than one sialidase domain. In cases in which a compound has more than one sialidase domain, the sialidase domains can be the same or different. Where the protein comprises multiple anchoring domains, the anchoring domains can be arranged in tandem (with or without linkers) or on alternate sides of other domains, such as sialidase domains. Where a compound comprises multiple sialidase domains, the sialidase domains can be arranged in tandem (with or without linkers) or on alternate sides of other domains.

### Sialidase Domain

The sialidase domain expressed by the oncolytic virus can be specific for Neu5Ac linked via alpha 2,3 linkage, specific for Neu5Ac linked via an alpha 2,6 or can cleave Neu5Ac linked via an alpha 2,3 linkage or an alpha 2,6 linkage. A variety of sialidases are described in Tables 2-5.

A sialidase that can cleave more than one type of linkage between a sialic acid residue and the remainder of a substrate molecule, in particular, a sialidase that can cleave both alpha(2, 6)-Gal and alpha(2, 3)-Gal linkages can be used in the compounds of the disclosure. Sialidases included are the large bacterial sialidases that can degrade the receptor sialic acids Neu5Ac alpha(2,6)-Gal and Neu5Ac alpha(2,3)-Gal. For example, the bacterial sialidase enzymes from Clostridium perfringens (Genbank Accession Number X87369), Actinomyces viscosus (GenBankX62276), Arthrobacter ureafaciens GenBank (AY934539), or Micromonospora viridifaciens (Genbank Accession Number D01045) can be used. Sialidase domains of compounds of the present disclosure can comprise all or a portion of the amino acid sequence of a large bacterial sialidase or can comprise amino acid sequences that are substantially homologous to all or a portion of the amino acid sequence of a large bacterial sialidase. In one preferred embodiment, a sialidase domain comprises a sialidase encoded by Actinomyces viscosus, such as that of SEQ ID NO: 1 or 2, or such as sialidase sequence substantially homologous to SEQ ID NO: 12. In yet another preferred embodiment, a sialidase domain comprises the catalytic domain of the Actinomyces viscosus sialidase extending from amino acids 274-666 of SEQ ID NO: or a substantially homologous sequence.

Additional sialidases include the human sialidases such as those encoded by the genes NEU2 (SEQ ID NO:8; Genbank Accession Number Y16535; Monti, E, Preti, Rossi, E., Ballabio, A and Borsani G. (1999) Genomics 57:137-143) and NEU4 (SEQ ID NO:9; Genbank Accession Number NM080741; Monti et al. (2002) Neurochem Res 27:646-663). Sialidase domains of compounds of the present disclosure can comprise all or a portion of the amino acid sequences of a sialidase or can comprise amino acid sequences that are substantially homologous to all or a portion of the amino acid sequences of a sialidase. Preferably, where a sialidase domain comprises a portion of the amino acid sequences of a naturally occurring sialidase, or sequences substantially homologous to a portion of the amino acid sequences of a naturally occurring sialidase, the portion comprises essentially the same activity as the intact sialidase. The present disclosure also includes sialidase catalytic domain proteins. As used herein a “sialidase catalytic domain protein” comprises a catalytic domain of a sialidase but does not comprise the entire amino acid sequence of the sialidase from which the catalytic domain is derived. A sialidase catalytic domain protein has sialidase activity. Preferably, a sialidase catalytic domain protein comprises at least 10%, at least 20%, at least 50%, at least 70% of the activity of the sialidase from which the catalytic domain sequence is derived. More preferably, a sialidase catalytic domain protein comprises at least 90% of the activity of the sialidase from which the catalytic domain sequence is derived.

A sialidase catalytic domain protein can include other amino acid sequences, such as but not limited to additional sialidase sequences, sequences derived from other proteins, or sequences that are not derived from sequences of naturally occurring proteins. Additional amino acid sequences can perform any of a number of functions, including contributing other activities to the catalytic domain protein, enhancing the expression, processing, folding, or stability of the sialidase catalytic domain protein, or even providing a desirable size or spacing of the protein.

A preferred sialidase catalytic domain protein is a protein that comprises the catalytic domain of the A. viscosus sialidase. Preferably, an A. viscosus sialidase catalytic domain protein comprises amino acids 270-666 of the A. viscosus sialidase sequence (SEQ ID NO:12). Preferably, an A. Viscosus sialidase catalytic domain protein comprises an amino acid sequence that begins at any of the amino acids from amino acid 270 to amino acid 290 of the A. viscosus sialidase sequence (SEQ ID NO: 12) and ends at any of the amino acids from amino acid 665 to amino acid 901 of said A. viscosus sialidase sequence (SEQ ID NO: 12), and lacks any A. viscosus sialidase protein sequence extending from amino acid 1 to amino acid 269.

In some preferred embodiments, an A. viscosus sialidase catalytic domain protein comprises amino acids 274-681 of the A. viscosus sialidase sequence (SEQ ID NO: 12) and lacks other A. viscosus sialidase sequence. In some preferred embodiments, an A. viscosus sialidase catalytic domain protein comprises amino acids 274-666 of the A. viscosus sialidase sequence (SEQ ID NO: 12) and lacks any other A. viscosus sialidase sequence. In some preferred embodiments, an A. viscosus sialidase catalytic domain protein comprises amino acids 290-666 of the A. viscosus sialidase sequence (SEQ ID NO: 12) and lacks any other A. viscosus sialidase sequence. In yet other preferred embodiments, an A. viscosus sialidase catalytic domain protein comprises amino acids 290-681 of the A. viscosus sialidase sequence (SEQ ID NO: 12) and lacks any other A. viscosus sialidase sequence.

TABLE 2
Engineered Sialidases

| Name | Sequence |
|---|---|
| AvCD | MGDHPQATPAPAPDASTELPASMSQAQHLAANTATDNYRIPAI |
| | TTAPNGDLLISYDERPKDNGNGGSDAPNPNHIVQRRSTDGGKT |
| | WSAPTYIHQGTETGKKVGYSDPSYVVDHQTGTIFNFHVKSYDQ |
| | GWGGSRGGTDPENRGIIQAEVSTSTDNGWTWTHRTITADITKD |
| | KPWTARFAASGQGIQIQHGPHAGRLVQQYTIRTAGGAVQAVSV |
| | YSDDHGKTWQAGTPIGTGMDENKVVELSDGSLMLNSRASDGSG |
| | FRKVAHSTDGGQTWSEPVSDKNLPDSVDNAQIIRAFPNAAPDD |
| | PRAKVLLLSHSPNPRPWSRDRGTISMSCDDGASWTTSKVFHEP |
| | FVGYTTIAVQSDGSIGLLSEDAHNGADYGGIWYRNFTMNWLGE |
| | QCGQKPAE |
| | (SEQ ID NO: 1) |
| DAS181 | MGDHPQATPAPAPDASTELPASMSQAQHLAANTATDNYRIPAI |
| | TTAPNGDLLISYDERPKDNGNGGSDAPNPNHIVQRRSTDGGKT |
| | WSAPTYIHQGTETGKKVGYSDPSYVVDHQTGTIFNFHVKSYDQ |
| | GWGGSRGGTDPENRGIIQAEVSTSTDNGWTWTHRTITADITKD |
| | KPWTARFAASGQGIQIQHGPHAGRLVQQYTIRTAGGAVQAVSV |
| | YSDDHGKTWQAGTPIGTGMDENKVVELSDGSLMLNSRASDGSG |
| | FRKVAHSTDGGQTWSEPVSDKNLPDSVDNAQIIRAFPNAAPDD |
| | PRAKVLLLSHSPNPRPWSRDRGTISMSCDDGASWTTSKVFHEP |
| | FVGYTTIAVQSDGSIGLLSEDAHNGADYGGIWYRNFTMNWLGE |
| | QCGQKPAKRKKKGGKNGKNRRNRKKKNP |
| | (SEQ ID NO: 2) |

TABLE 3
Human Sialidases

| | Uniprot | |
|---|---|---|
| Name | Identifier | SEQ ID NO |
| Human Neu 1 | Q99519 | 3 |
| Human Neu 2 | Q9Y3R4 | 4 |
| Human Neu 3 | Q9UQ49 | 5 |
| Human Neu 4 | Q8WWR8 | 6 |
| Human Neu 4 | Q8WWR8 | 7 |
| Isoform 2 | | |
| Human Neu 4 | Q8WWR8 | 8 |
| Isoform 3 | | |

TABLE 4
Sialidases in organisms that are
largely commensal with humans

| | Uniprot/ | | |
|---|---|---|---|
| | Genbank | Gene | SEQ |
| Organism | ID | name | ID NO |
| Actinomyces viscosus | Q59164 | nanH | 9 |
| Actinomyces viscosus | A0A448PLN7 | nanA | 10 |
| Streptococcus oralis | A0A081R4G6 | nanA | 11 |
| Streptococcus oralis | D4FUA3 | nanH | 12 |
| Streptococcus mitis | A0A081Q0I6 | nanA | 13 |
| Streptococcus mitis | A0A3R9LET9 | nanA_1 | 14 |
| Streptococcus mitis | A0A3R9J1C3 | nanA_2 | 15 |
| Streptococcus mitis | A0A3R9IIK2 | nanA_3 | 16 |
| Streptococcus mitis | A0A3R9IXG7 | nanA_4 | 17 |
| Streptococcus mitis | A0A3R9K5C5 | nanA_5 | 18 |
| Streptococcus mitis | J1H2U0 | nanH | 19 |
| Porphyromonas gingivalis | B2RL82 | | 20 |
| Tannerella forsythia | Q84BM9 | siaHI | 21 |
| Tannerella forsythia | A0A1D3USB1 | nanH | 22 |
| Akkermansia Muciniphila | B2UPI5 | | 23 |
| Akkermansia Muciniphila | B2UN42 | | 24 |
| Bacteroides thetaiotaomicron | Q8AAK9 | | 25 |

TABLE 5
Additional sialidases

| | Uniprot/Genbank |
|---|---|
| Organism | ID |
| Actinotignum schaalii | S2VK03 |
| Anaerotruncus colihominis | B0PE27 |
| Ruminococcus gnavus | A0A2N5NZH2 |
| Clostridium difficile | Q185B3 |
| Clostridium septicum | P29767 |
| Clostridium perfringens | P10481 |
| Clostridium perfringens | Q8XMY5 |
| Clostridium perfringens | A0A2Z3TZA2 |
| Vibrio cholerae | P0C6E9 |
| Salmonella typhimurium | P29768 |
| Paeniclostridium sordellii | A0A446I8A2 |
| Streptococcus pneumoniae (NanA) | P62576 |
| Streptococcus pneumoniae (NanB) | Q54727 |
| Pseudomonas aeruginosa | A0A2X4HZU8 |
| Aspergillus fumigatus | Q4WQS0 |
| Arthrobacter ureafaciens | Q5W7Q2 |
| Micromonospora viridifaciens | Q02834 |

### Anchoring Domain

As used herein, an “extracellular anchoring domain” or “anchoring domain” is any moiety that interacts with an entity that is at or on the exterior surface of a target cell or is in close proximity to the exterior surface of a target cell. An anchoring domain serves to retain a compound of the present disclosure at or near the external surface of a target cell. An extracellular anchoring domain preferably binds 1) a molecule expressed on the surface of a cancer cell, or a moiety, domain, or epitope of a molecule expressed on the surface of a cancer cell, 2) a chemical entity attached to a molecule expressed on the surface of a cancer cell, or 3) a molecule of the extracellular matrix surrounding a cancer cell.

Useful anchoring domains bind to heparin/sulfate, a type of GAG that is ubiquitously present on cell membranes. Many proteins specifically bind to heparin/heparan sulfate, and the GAG-binding sequences in these proteins have been identified (Meyer, F A, King, M and Gelman, R A. (1975) Biochimica et BiophysicaActa 392: 223-232; Schauer, S. ed., pp 233. Sialic Acids Chemistry, Metabolism and Function. Springer-Verlag, 1982). For example, the GAG-binding sequences of human platelet factor 4 (PF4) (SEQ ID NO:2), human interleukin 8 (IL8) (SEQ ID NO:3), humanantithrombin III (AT III) (SEQ ID NO:4), human apoprotein E (ApoE) (SEQ ID NO:5), human angio-associated migratory cell protein (AAMP) (SEQ ID NO:6), or human amphiregulin (SEQ ID NO:7) have been shown to have very high affinity to heparin.

### Linkers

A protein that includes a sialidase or a catalytic domain thereof can optionally include one or more polypeptide linkers that can join domains of the compound. Linkers can be used to provide optimal spacing or folding of the domains of a protein. The domains of a protein joined by linkers can be sialidase domains, anchoring domains, or any other domains or moieties of the compound that provide additional functions such as enhancing protein stability, facilitating purification, etc. Some preferred linkers include the amino acid glycine. For example, linkers having the sequence: (GGGGS (SEQ ID NO:10))n, where n is 1-20.

### EXAMPLES

### Example 1: DAS181 Treatment Reduces Surface Sialic Acid on Tumor Cells

In this study the impact of DAS181 on the sialic acid burden of certain tumor cells was examined. Briefly, FACs and image-based quantitation of α-2,3 and α-2,6 sialic acid modifications on A549 (human alveolar basal epithelial adenocarcinoma) and MCF (human mamillary epithelial adenocarcinoma) tumor cells were conducted. Galatose exposure after sialic acid removal in A549 and MCF7 cells was detected by PNA-FITC using flow cytometry analysis and imaging approaches. As discussed above, there are two sialic acid is most often attached to the penultimate sugar by an α-2,3 linkage or an α-2,6 linkage, which can that can be detected by Maackia Amurensis Lectin II (MAL II) and Sambucus Nigra Lectin (SNA), respectively. In addition, surface galactose (e.g., galactose exposed after sialic acid removal) can be detected using Peanut Agglutinin (PNA).

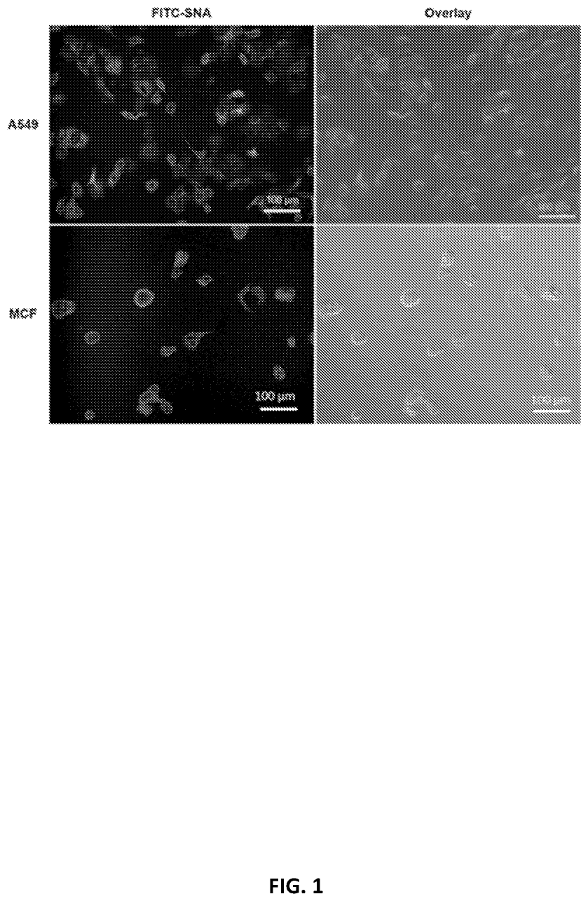

FIG. 1 depicts the detection of 2,6 sialic acid by FITC-SNA on A549 and MCF cells by fluorescence imaging.

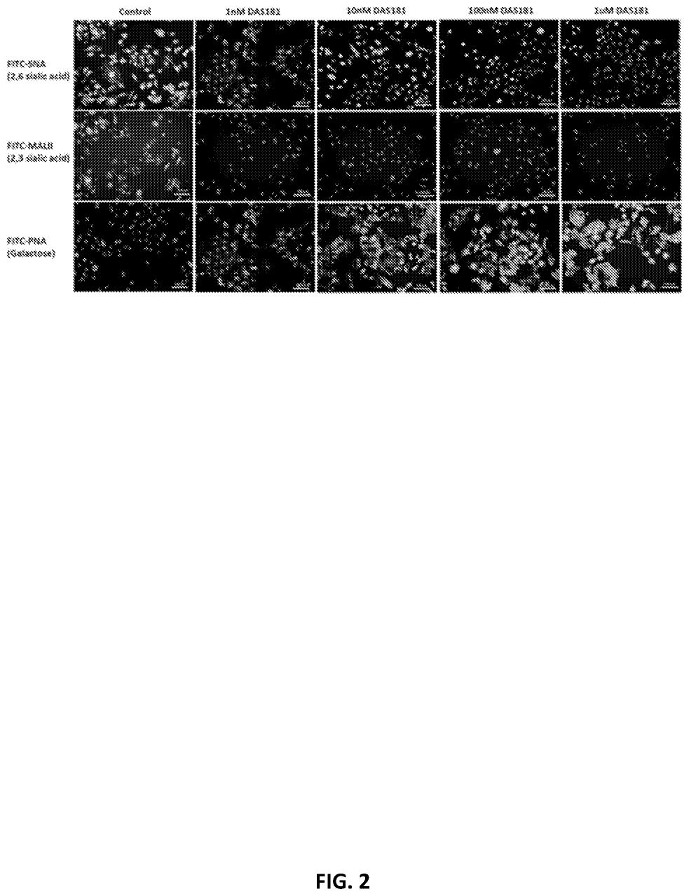

A549 cells were treated with various concentrations of DAS181 and them stained to image 2,6 linked sialic acid (FITC-SNA), 2,3 linked sialic acid (FITC-MALII) or galactose (FITC-PNA). As can be seen in FIG. 2, DAS181 effectively removed both 2,3 and 2,6 linked sialic acid and exposed galactose.

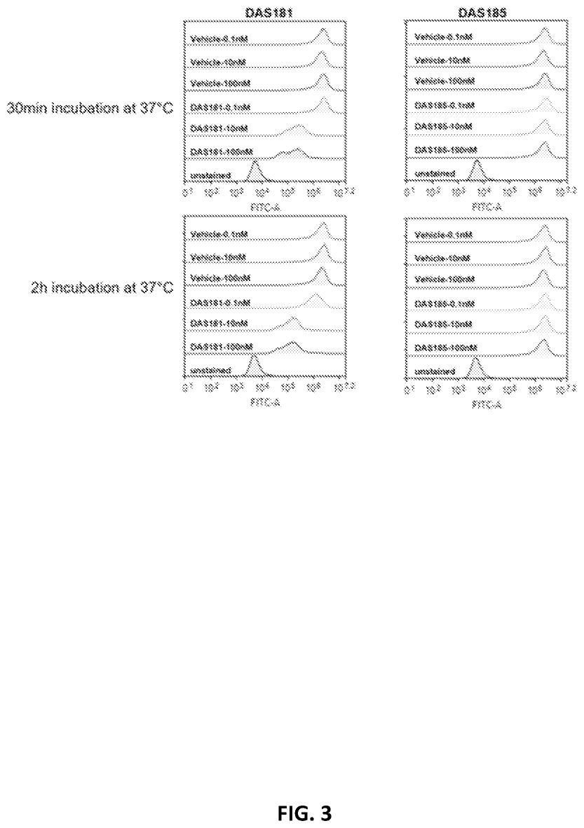

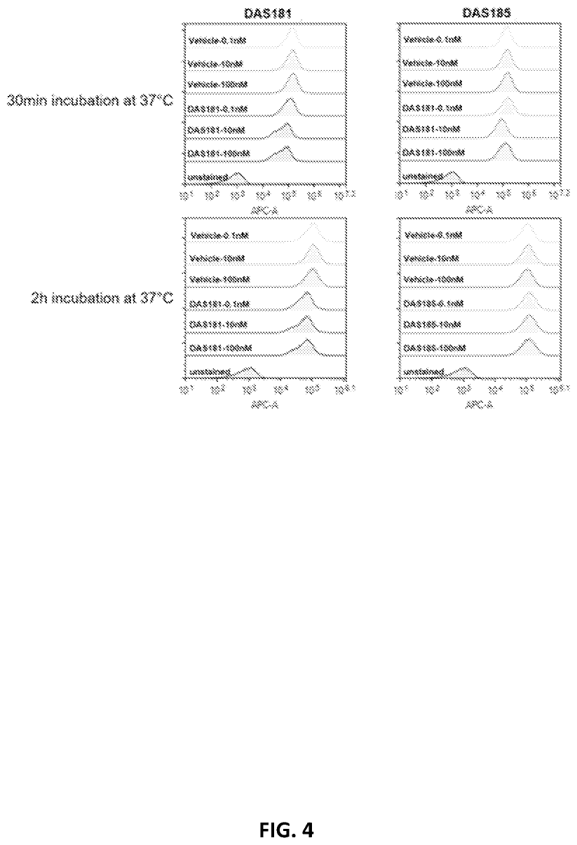

In contrast, DAS185, a variant of DAS181 lacking sialidase activity due to Y348F mutation, was not able to remove 2,6 linked sialic acid or 2,3 linked sialic acid. As shown in FIG. 3, incubation of A549 cells with DAS185 had essentially no impact on surface 2,3 linked sialic acid, while DAS181 reduced surface 2,3 linked sialic acid in a concentration dependent manner. Similarly, incubation of A549 cells with DAS185 had essentially no impact on surface 2,6 linked sialic acid, while DAS181 reduced surface 2,6 linked sialic acid in a concentration dependent manner (FIG. 4). Consistent with these results, incubation of A549 cells with DAS185 had essentially no impact on surface galactose, while DAS181 increased surface galactose in a concentration dependent manner.

### Example 2: DAS181 Treatment Increases PDMC-Mediated Tumor Cell Killing

A549 cells were genetically labelled with a red fluorescent protein (A549-red). Fresh human PMBCs were harvested and stimulated with various cytokine and antibody combinations to activate effector T cells (CD3, CD38 and IL-2) or, in some cases, T cells and NK cells (CD3, CD28, IL-15 and IL-21). Activated PBMCs were then co-cultured with A549-red cells that had been exposed to DAS181 (100 nM). Tumor cell killing by PBMCs was monitored by live cell imaging and quantification with IncuCyte. The cell culture medium was collected and analyzed by ELISA to assess cytokine production by PBMCs.

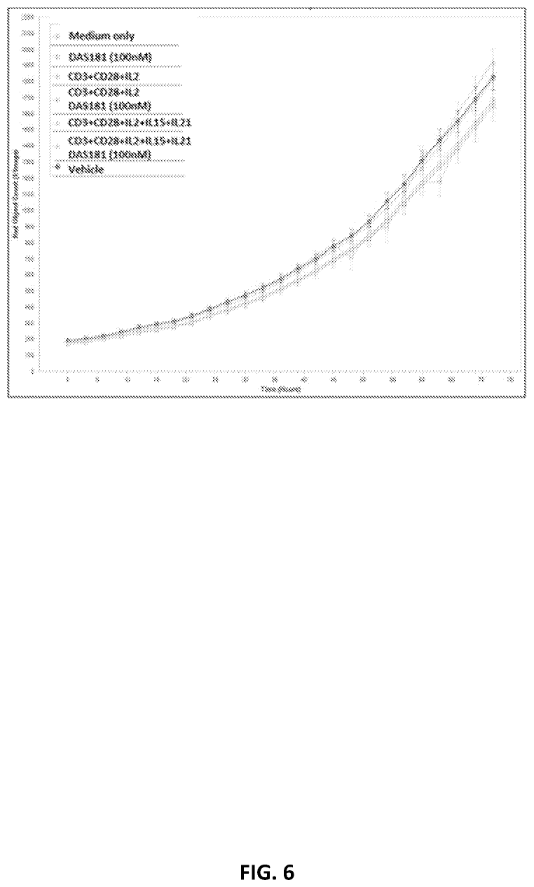

FIG. 6 shows that neither the treatments used to stimulate PBMC nor DAS181 in combination with treatment used to stimulate PBMC impact A549-red cell proliferation.

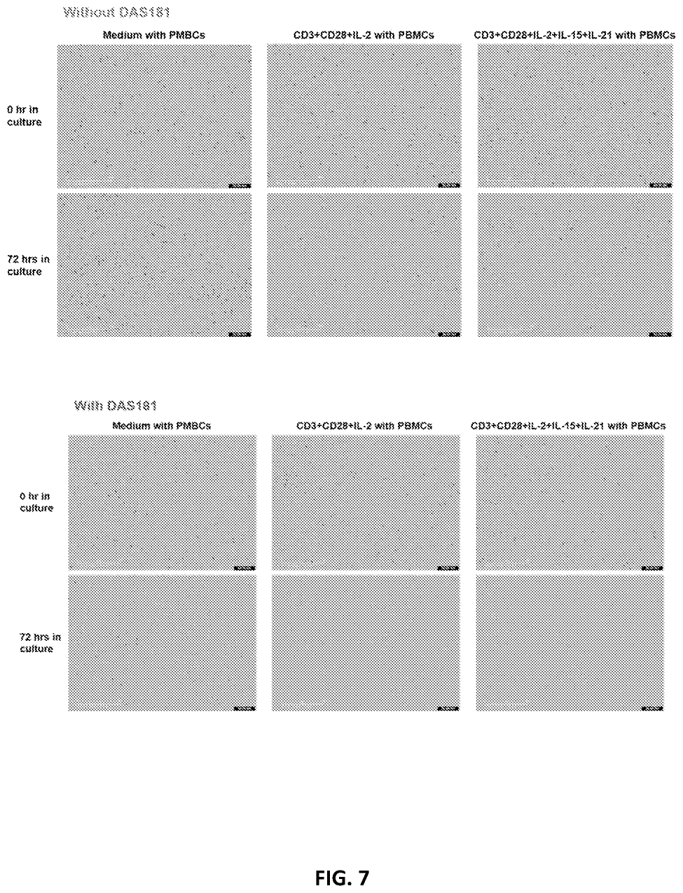

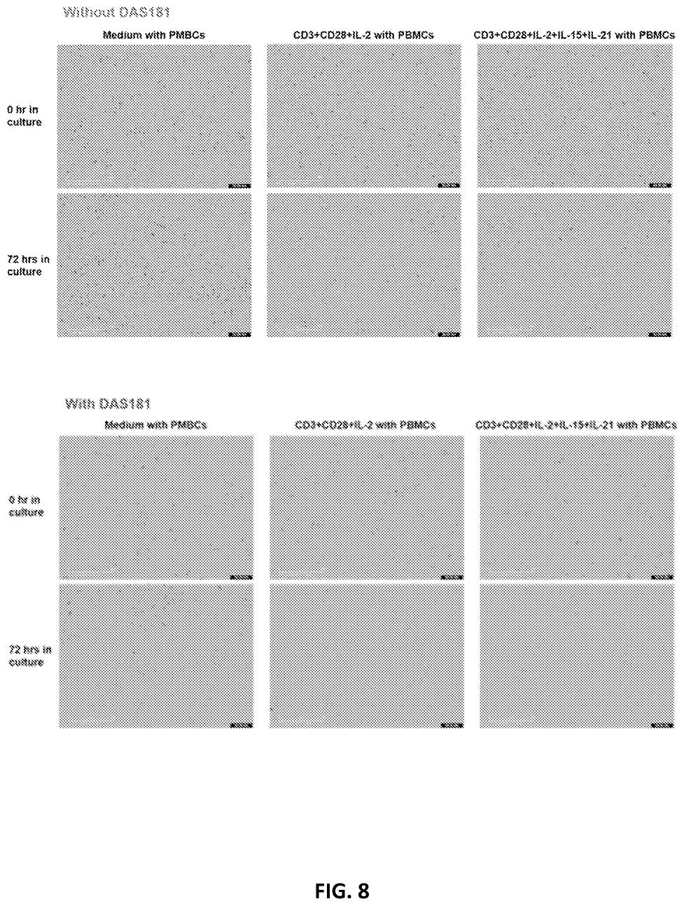

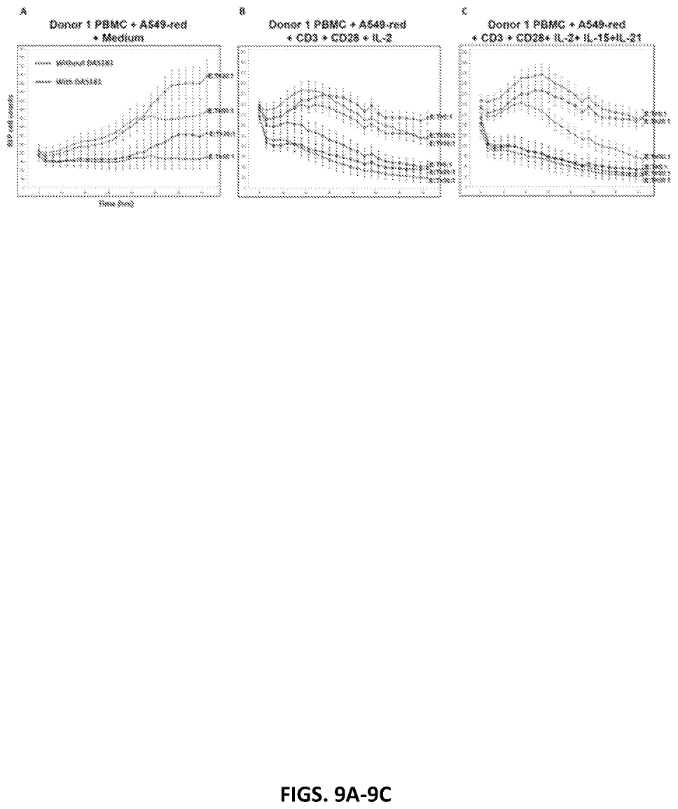

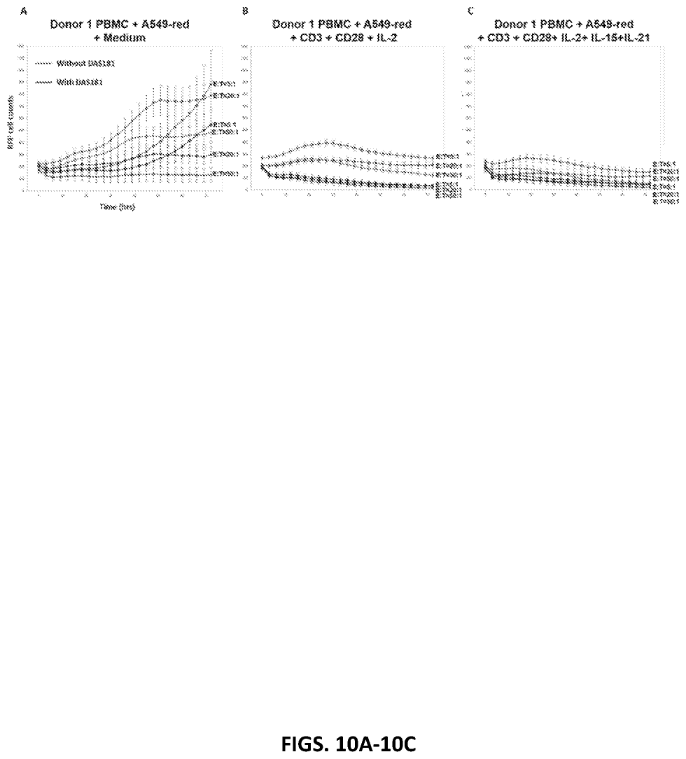

FIG. 7 shows that DAS181 significantly increases tumor cell toxicity mediated by PBMC (Donor 1), both T cell mediated and NK cell mediated, compared to a vehicle only control. Similar results were observed using PBMC from a different donor (Donor 2; FIG. 8). FIG. 9 and FIG. 10 present a quantification of the data presented in FIG. 7 and FIG. 8, respectively.

### Example 3: NK Cell Mediated Killing of Tumor Cells by Oncolytic Vaccinia Virus and DAS181

In this study the impact of an oncolytic vaccina virus (Western Reserve) and DAS181 on NK cell-mediated killing was examined. DAS185, a variant protein lacking sialidase activity was used as a control.

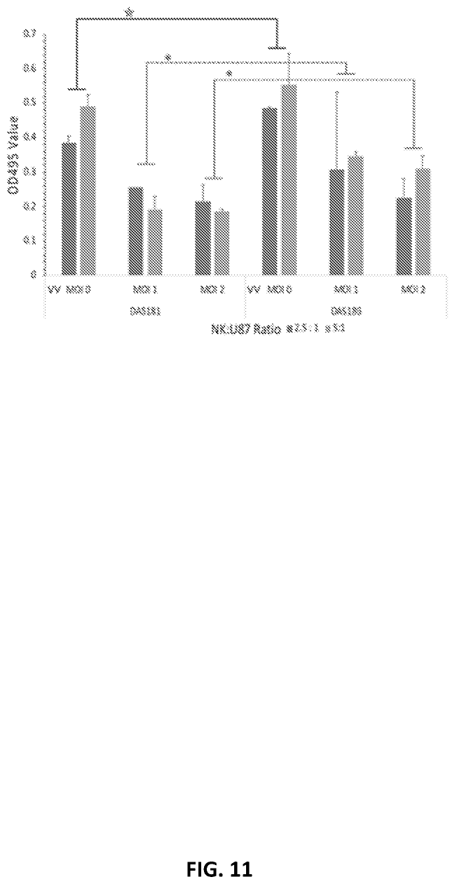

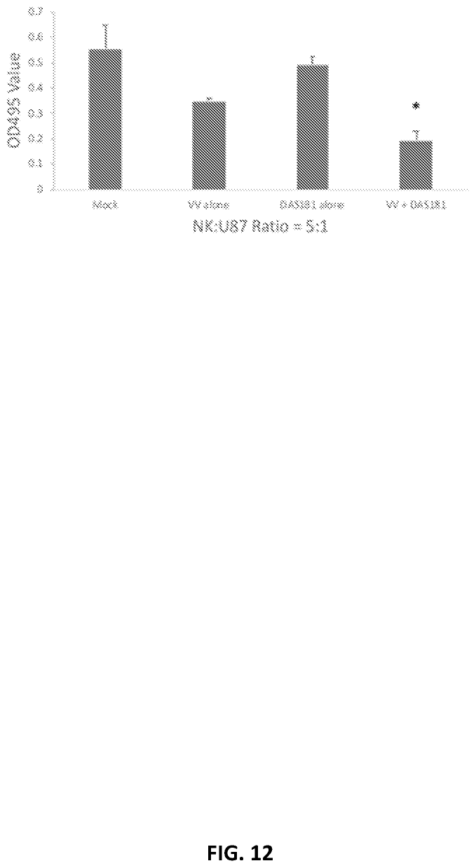

Briefly, tumor cells (U87-GFP) were plated in a 96-well tissue culture plate at 5×10⁴ cells per well (100 ul) in DMEM and incubated overnight at 37° C. On Day 2 the cells were infected with VV at MOI 0.5, 1, or 2 in fetal bovine serum-free medium for 2 hours and then exposed to 1 nM DAS181 or 1 mM DAS185. Tumor cells were then mixed with purified NK cells at Effector:Tumor (E:T)=1:1, 5:1, 10:1. The cells were cultured in medium supplemented with 2% FBS in order to decrease neuraminidase/sialidase background. After 24 hrs, tumor killing were measured by MTS assay (96 well plate), and cell culture medium was collected. Expression of IFN gamma were measured by ELISA. The results of this study are shown in FIG. 11 and FIG. 12 where it can be seen the DAS181, but not inactive DAS185, increased tumor cell killing by oncolytic vaccinia virus.

### Example 4: Impact of DAS181 on DC Maturation and Antigen Presenting Activity in the Presence of Tumor Cells

In this study, the impact of DAS181 on monocyte-derived dendritic cell was examined DAS185, a variant protein lacking sialidase activity was used as a control.

Briefly, monocyte-derived dendritic cells (DC) were prepared by resuspending 5×10⁶ adherent PBMC in 3 ml of medium supplemented with 100 ng/ml of GM-CSF and 50 ng/ml of IL-4. After 48 hrs, 2 ml of fresh medium supplemented with 100 ng/ml of GM-CSF and 50 ng/ml of IL-4 was added to each well. After another 72 hrs, tumor cell (U87-GFP) were plated in 24-well plates in DMEM. The tumor cells were infected with VV at various MOI in FBS free medium for 2 hours. DC cultured in the presence of 1 nM DAS181 or DAS185 were mixed with tumor cells at 1:1 tumor cell:DC ratio. Dendritic cell maturation (expression of CD86, CD80, MHC-I) and production of pro-inflammatory cytokines (TNF-alpha) was then measured and quantified by flow cytometry and ELISA, respectively.

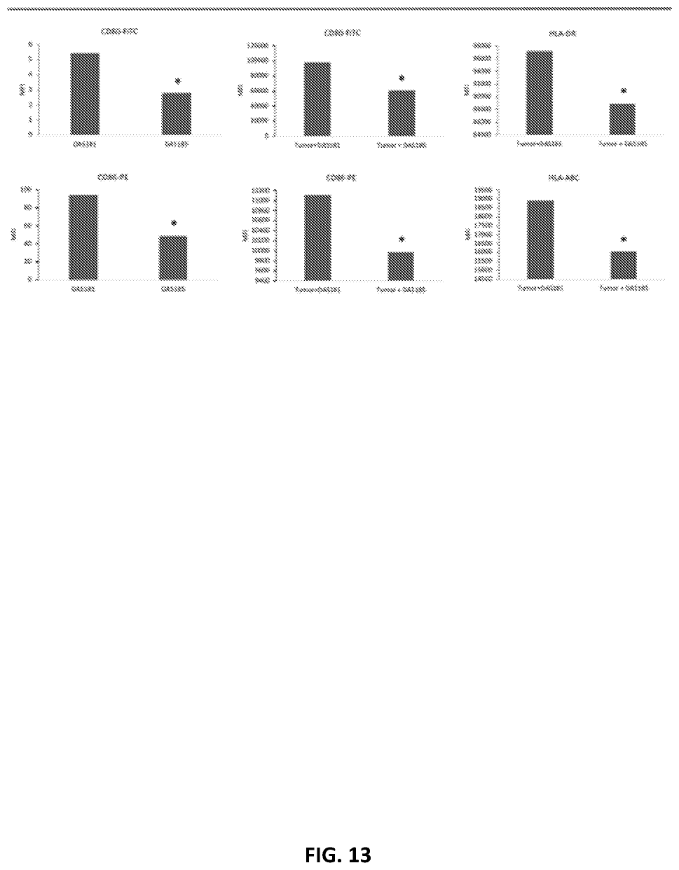

As can be seen in FIG. 13, DAS181 significant enhanced expression of dendritic cell maturation markers whether the cells were cultured alone or with vaccinia virus infected tumor cells.

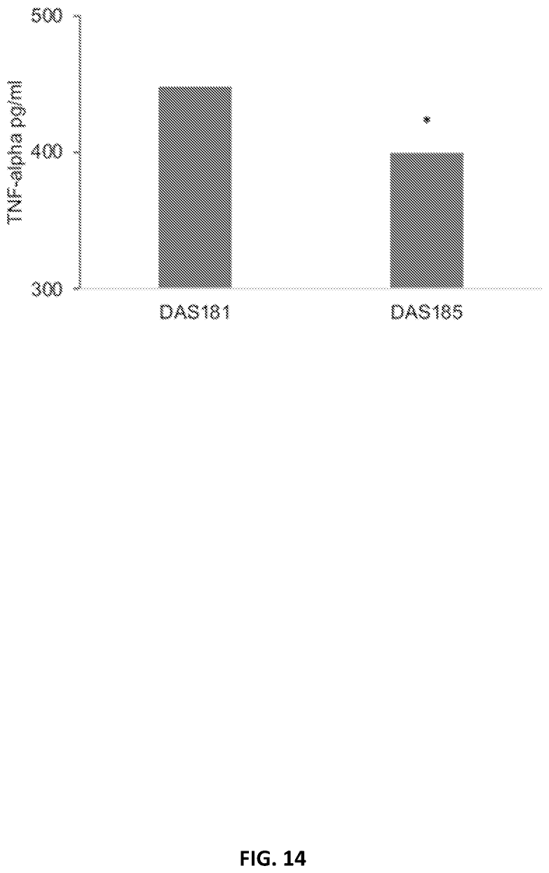

The results of this study demonstrate that exposure to DAS181 increased and increased TNF-alpha secretion by dendritic cells (FIG. 14).

### Example 5: DAS181 Increases Oncolytic Adenovirus Tumor Cell Killing in the Absence of Immune Cells

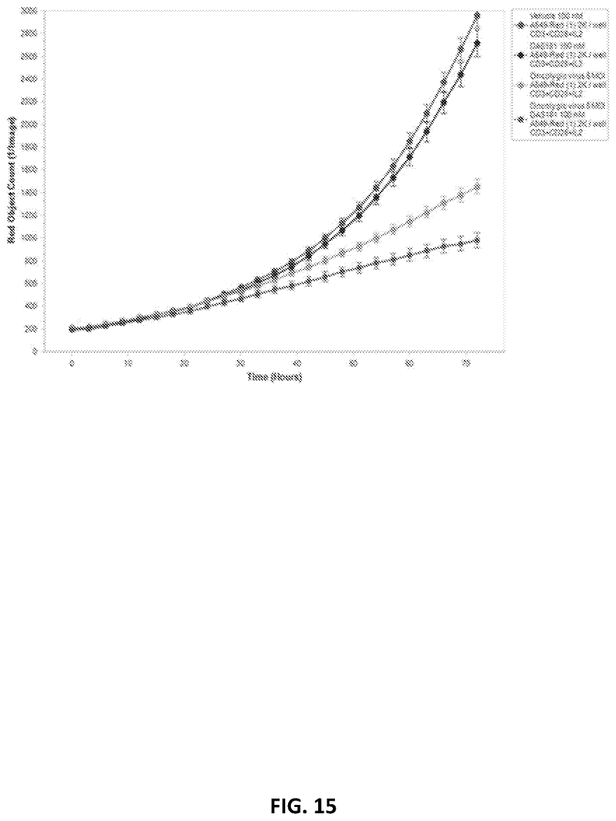

A549 cells were genetically labelled with red fluorescent protein (A549-red). Tumor cell proliferation and killing by oncolytic adenovirus (Ad5) in the presence or absence of DAS181 was monitored by live cell imaging and quantification with IncuCyte. The cell culture medium was collected for ELISA measurement of cytokine production by PBMCs. As shown in FIG. 15, DAS181 increased oncolytic adenovirus-mediated tumor cell killing and growth inhibition.

### Example 6: DAS181 Increases Oncolytic Adenovirus Tumor Cell Killing in the Presence of PBMC

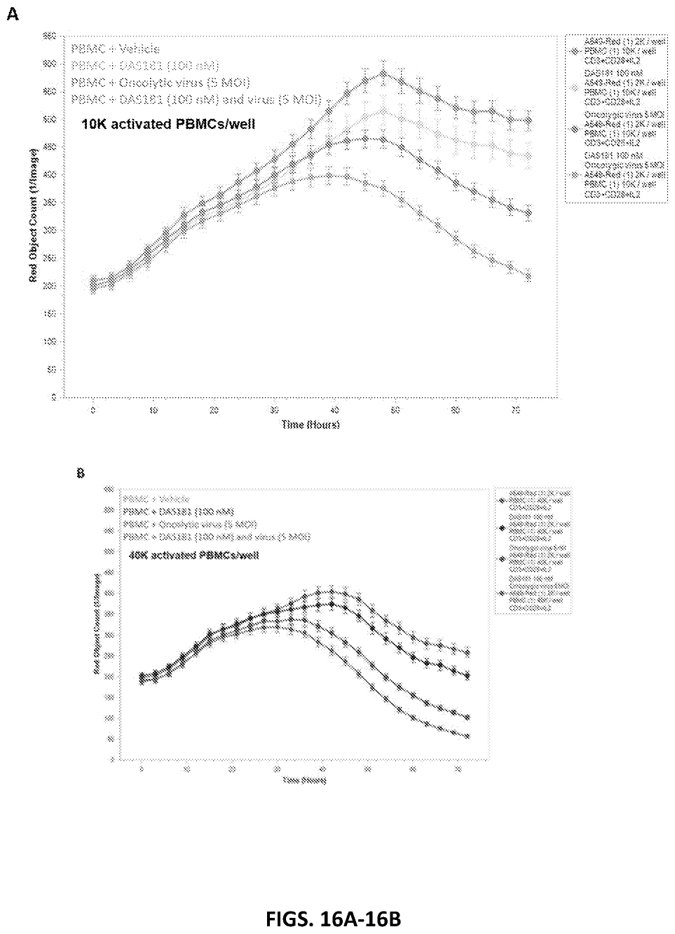

A549 cells were genetically labelled by a red fluorescent protein (A549-red). Fresh human PMBCs were harvested and stimulated with proper cytokine and antibody combinations to activate effector T cells. Activated PBMCs were then co-cultured with A549-red cells that have been treated with DAS181 with or without the oncolytic adenovirus (Ad5). Tumor cell killing by PBMCs was monitored by live cell imaging and quantification with IncuCyte. The cell culture medium was collected for ELISA measurement of cytokine production by PBMCs. As shown in FIG. 16, DAS181 significantly increased tumor cell killing when present together with oncolytic adenovirus in the presence of PBMC.

### Example 7: Construction and Characterization of an Oncolytic Virus Expressing DAS181

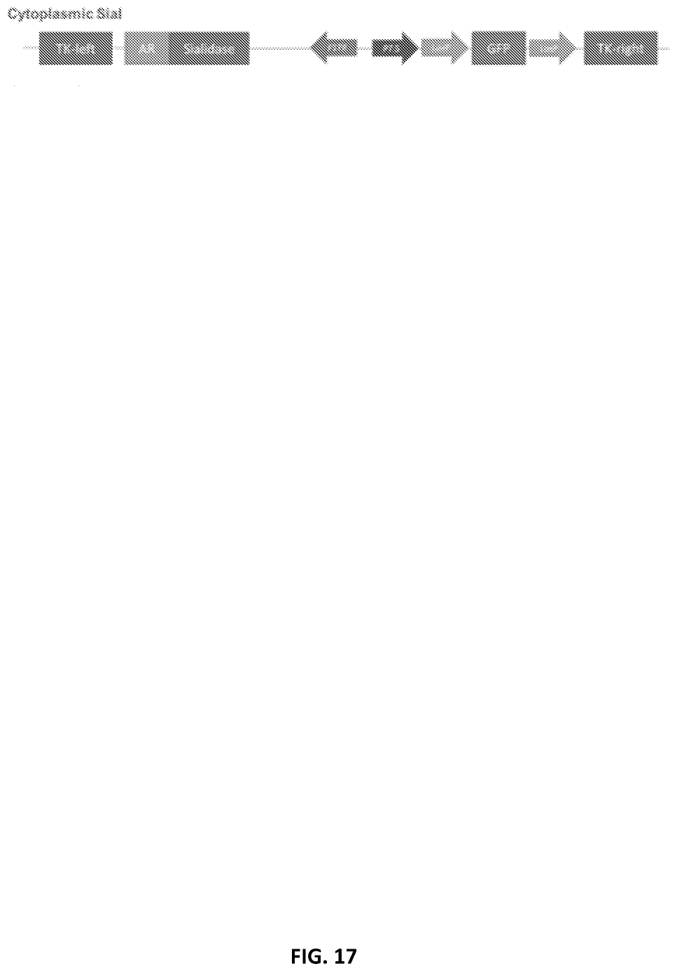

A construct designed for expression of DAS181 is depicted schematically in FIG. 17.

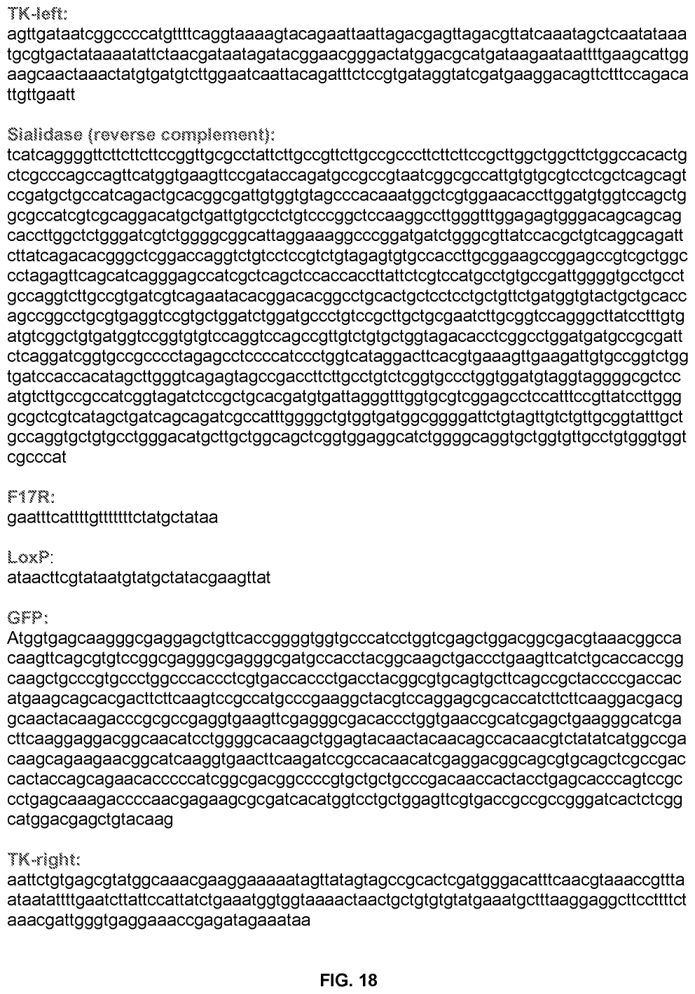

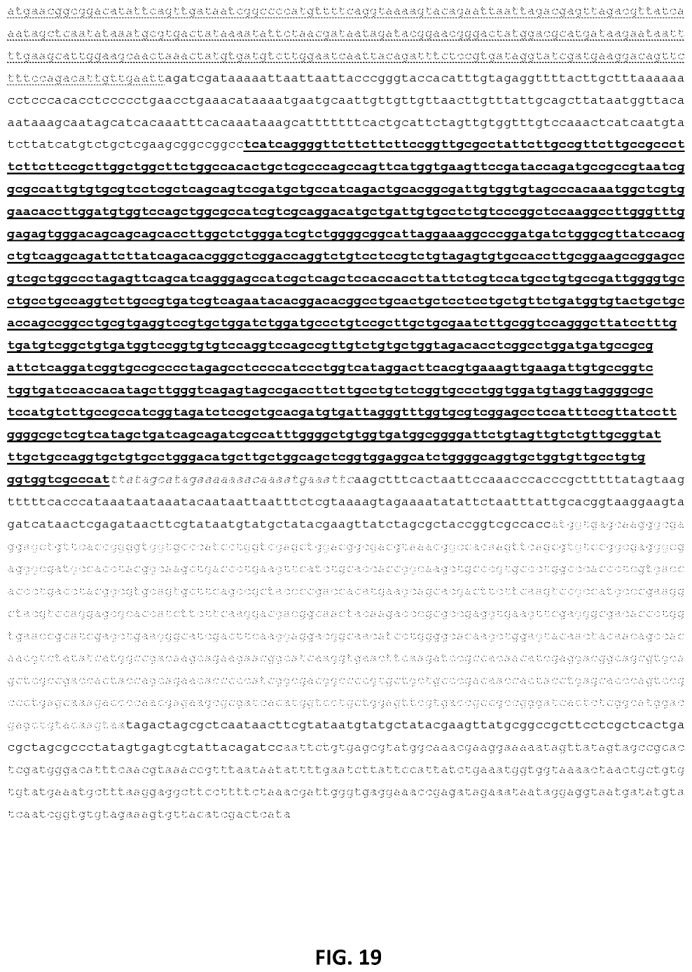

To generate a recombinant VV expressing DAS181, a pSEM-1 vector was modified to include a sequence encoding DAS181 as well as two loxP sites with the same orientation flanking the sequence encoding the GFP protein (pSEM-1-TK-DAS181-GFP). DAS181 expression is under the transcriptional control of the F17R late promoter in order to limit the expression within tumor tissue. The sequences certain of the components and a portion of the construct and are shown in FIG. 18 and FIG. 19.

Western Reserve VV was used as the parental virus. VV expressing DAS181 was generated by recombination with pSEM-1-TK-DAS181-GFP into the TK gene of Western Reserve VV to generated VV-DAS181.

Recombinant Virus can be Generated as Follows.

### Transfection:

Seed CV-1 cells in 6-well plate at 5×10⁵ cells/2 ml DMEM-10% FBS/well and grow overnight. Prepare parent VV virus (1 ml/well) by diluting a virus stock in DMEM/2% FBS at MOI 0.05. Remove medium from CV-1 wells and immediately add VV, and culture for 1-2 hours. CV-1 cells should be 60-80% confluent at this point. Transfection mix in 1.5 ml tubes. For each Transfection, dilute 9 ul Genejuice in 91 ul serum-free DMEM and incubate at room temperature for 5 min. Add 3 ug pSEM-1-TK-DAS181-GFP DNA gently by pipetting up and down two or three times. Leave at room temperature for 15 min. Aspirate VV virus from the CV-1 well and wash the cells once with 2 ml serum-free DMEM. Add 2 ml DMEM-2% FBS and add the DNA-genejuice solution drop-by-drop. Incubate at 37° C. for 48-72 hr or until all the cells round up. Harvest the cells by pipetting repeatedly. Release the virus from cells by repeated freeze-thawing of the harvested cells by first placing them in dry-ice/ethanol bath and then thawing them in a 37° C. water bath and vortexing. Repeat the freeze-thaw cycling three times. The cell lysate can be stored at −80° C.

### Plaque Isolation:

Seed CV-1 cells in 6-well plates at 5×10⁵ cells/2 ml DMEM-10% FBS/well and grow overnight. CV-1 cells should be 60-80% confluent when receiving cell lysate. Sonicate the cell lysate on ice using sonic dismembrator with an ultrasonic convertor probe for 4 cycles of 30 s until the material in the suspension is dispersed. Make 10-fold serial dilutions of the cell lysate in DMEM-2% FBS. Add 1 ml of the cell lysate-medium per well at dilutions 10⁻², 10⁻³, 10⁻⁴, incubate at 37° C. Pick well-separated GFP+ plaques using pipet tip. Rock the pipet tip slightly to scrape and detach cells in the plaque. Gently transfer to a microcentrifuge tube containing 0.5 ml DMEM medium. Freeze-thaw three times and sonicate. Repeat the same process of plaque isolation 3-5 times.

### Virus Amplification:

Seed CV-1 cells 5×10⁵ cells/2 ml DMEM-10% FBS/well and grow overnight in 6-well plate. CV-1 should be confluent when starting the experiment. Infect 1 well with 250 ul of plaque lysate/1 ml DMEM-2% FBS, and incubate at 37° C. for 2 h. Remove the plaque lysate and add 2 ml fresh DMEM-2% FBS, and incubate for 48-72 hr until cells round up. Collect the cells by repeatedly pipetting, freeze-thaw 3 times and sonicate. Add half of the cell lysate in 4 ml DMEM-2% FBS and infect CV-1 cells in 75-CM2 flask, after 2 h, remove virus and add 12 ml DMEM-2% FBS and culture 48-72 h (until cell round up). Harvest the cells, spin down 5 min at 1800 G, and discard supernatant and resuspend in 1 ml DMEM-2.5% FBS.

### Virus Titration:

Seed CV-1 cells 5×10⁵ cells/2 ml DMEM-10% FBS/well and grow overnight in 6-well plate. Dilute virus in DMEM-2% FBS, 50 ul virus/4950 ul DMEM-2% FBS (A, 10⁻²), 500 ul A/4500 ul medium (B, 10⁻³), and 500 ul B/4500 ul medium (C, 10⁻⁴), 10⁻⁷ to 10⁻¹⁰ for virus stock. Remove medium and wash 1× with PBS, and cells were infected with 1 ml virus dilution in duplicate. Incubate the cells for 1 h, rock the plate every 10 min. 1 h later, remove the virus and add 2 ml DMEM-10% FBS and incubate 48 h. Remove the medium, add 1 ml of 0.1% crystal violet in 20% ethanol for 15 min at room temperature. Remove the medium and allow to dry at room temperature for 24 hr. Count the plaque and express as plaque forming units (pfu) per ml.

### Detection of DAS181 Expression by VV-RAS181:

CV-1 cells were infected with VV-DAS181 at MOI 0.2. 48 hours later, CV-1 cells were collected. DNA was extracted using Wizard SV Genomic DAN Purification System and used as template for DAS181 PCR amplification. PCR was conducted using standard PCR protocol and primer sequences (SialF: GGCGACCACCCACAGGCAACACCAGCACCTGCCCCA and SialR: CCGGTTGCGCCTATTCTTGCCGTTCTTGCCGCC). The expected PCR product (1251 bp) was found.

### Example 8: DAS181 Expressed by Vaccinia Virus is Active In Vitro

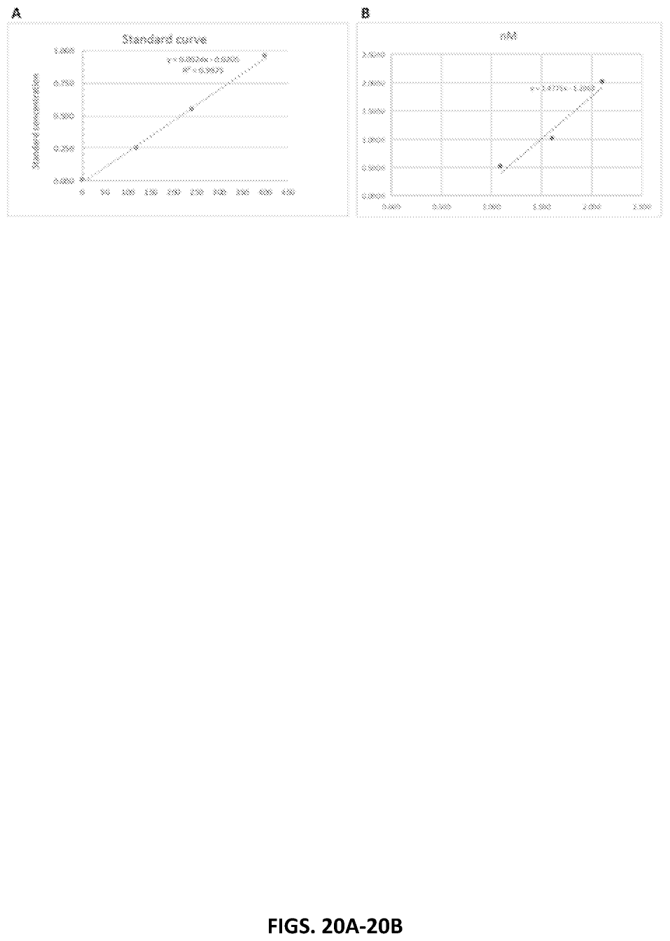

CV-1 cells were plated in six well plate. The cells were transduced with Sialidase-VV or control VV at MOI 0.1 or MOI 1. After 24 hrs, transfected cells were collected, and single cell suspension were made in PBS at 3×10⁶/500 ul. Cell lysate was prepared using Sigma's Mammalian cell lysis kit for protein extraction (Sigma, MCL1-1KT), and supernatant was collected. The sialidase (DAS181) activity was measured using Neuraminidase Assay Kit (Abcam, ab138888) according to manufacturer's instruction. 1 nM, 2 nM, and 10 nM DAS181 was added to the VV-cell lysate as control and generated the standard curve. 1×10\hat{ }6 cells infected with Sialidase-VV express DAS181 equivalent to 0.78 nM-1.21 nM of DAS181 in 1 ml medium. As shown in FIG. 20, the DAS181 has sialidase activity in vitro.

### Example 9: Vaccinia Virus-Sialidase Promotes Dendritic Cell Maturation

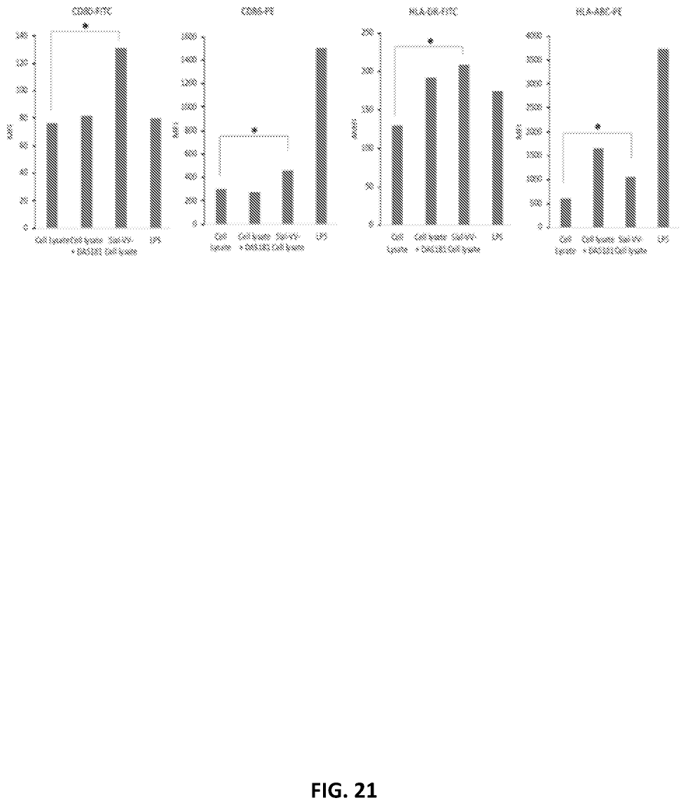

To determine if Sialidase-VV can promote DC activation and maturation, adherent human PBMC were re-suspend at 5×10⁶ cells in 3 ml medium supplemented with 100 ng/ml of GM-CSF and 50 ng/ml of IL-4 then cultured in 6-well plates with 2 ml per well of fresh medium supplemented with same concentrations of GM-CSF and IL-4. After 48 hrs, the cells were cultured in the presence of Sialidase-VV infected tumor cell lysate, VV-infected tumor cell lysate, VV-infected tumor cell lysate plus synthetic DAS181 protein, or LPS (positive control). After another 24 hrs, expression of CD86, CD80, MHC-II, MHC-I were determined by flow cytometry. As shown in FIG. 21, Sialidase-VV promotes the expression of markers indicative of dendritic cell activation and maturation.

### Example 10: Sialidase-VV Enhances T Lymphocyte-Mediated Cytokine Production and Oncolytic Activity

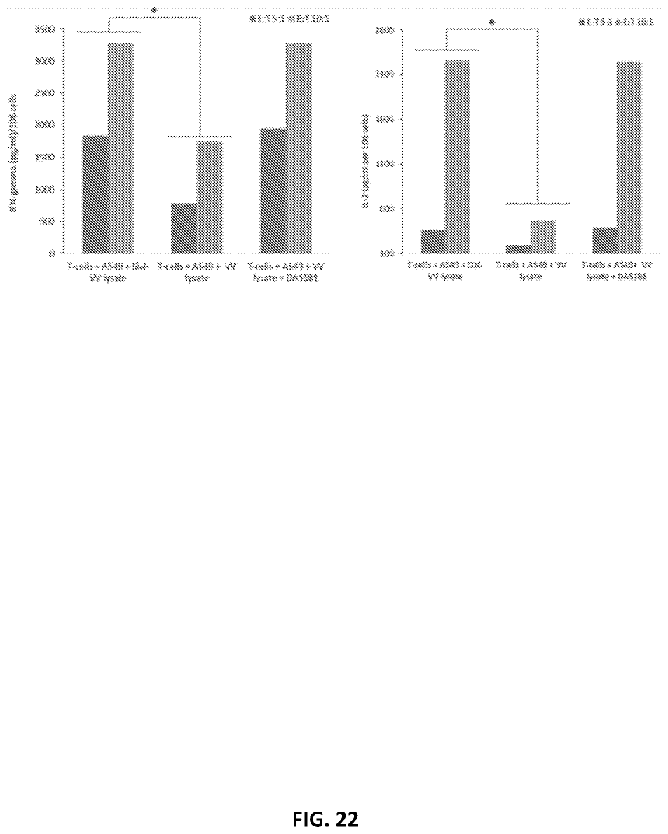

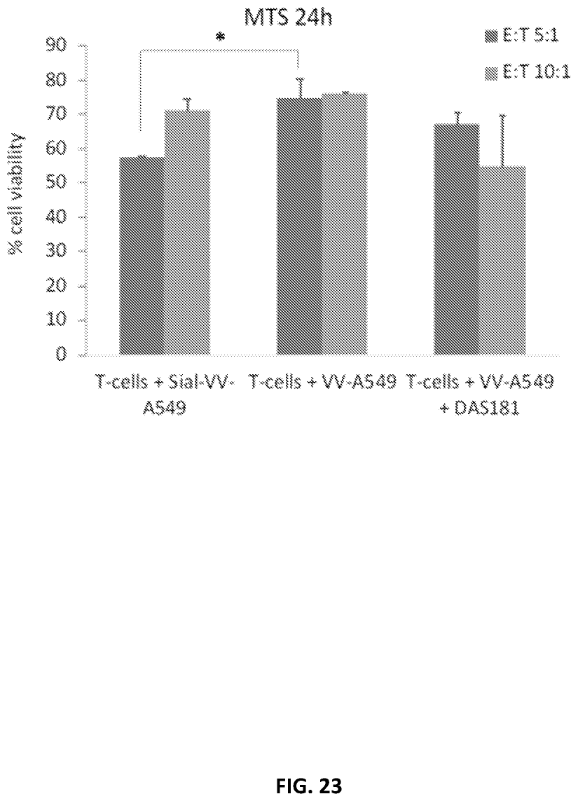

To assess whether DAS181 can activate human T cells by inducing IFN-gamma (IFNr) and IL-2 expressing, human PBMCs were activated by adding CD3 antibody at 10 ug/ml, proliferation was further stimulated by adding IL-2 by every 48 hrs. On day 15, tumor cells (A549) were infected with VVs at MOI 0.5, 1, or 2 in 2.5% FBS medium for 2 hours. Activated T cells were added to the culture at effector:target ratio of 5:1 or 10:1 in the presence of CD3 antibody at 1 ug/ml. After another 24 hrs, tumor cytotoxicity was measured and cell culture medium was collected for cytokine array. As can be seen in FIG. 22, Sialidase-VV induces a significantly greater IL2 and IFN-gamma expression by CD3 activated T cells than does VV. In addition, as can be seen in FIG. 23, Sialidase-VV elicits stronger anti-tumor response than VV at and E;T of 5:1.

Sequences of certain Sialidases

```
SEQ ID NO: 3
                10 20 30 40 50 60 70 80
MTGERPSTAL PDRRWGPRIL GFWGGCRVWV FAAIFLLLSL AASWSKAEND FGLVQPLVTM EQLLWVSGRQ IGSVDTFRIP
                90 100 110 120 130 140 150 160
LITATPRGTL LAFAEARKMS SSDEGAKFIA LRRSMDQGST WSPTAFIVND GDVPDGLNLG AVVSDVETGV VFLVYSLCAH
              170 180 190 200 210 220 230 240
KAGCQVASTM LWVSKKDGVS WSTPRNLSLD IGTEVFAPGP GSGIQKQREP RKGRLIVCGH GTLERDGVFC LLSDDHGASW
              250 260 270 280 290 300 310 320
RYGSGVSGIP YGQPKQENDF NPDECQPYEL PDGSVVINAR NQNNYHCHCR IVLRSYDACD TLRPRDVTFD PELVDPVVAA
              330 340 350 360 370 380 390 400
GAVVTSSGIV FFSNPAHPEF RVNLTLRWSF SNGTSWRKET VQLWPGPSGY SSLATLEGSM DGEEQAPQLY VLYEKGRNHY
              410
TESISVAKIS VYGTL
SEQ ID NO: 4
                10 20 30 40 50 60 70 80
MASLPVLQKE SVFQSGAHAY RIPALLYLPG QQSLLAFAEQ RASKKDEHAE LIVLRRGDYD APTHQVQWQA QEVVAQRALD
                90 100 110 120 130 140 150 160
GHRSMNPCPL YDAQTGTLFL FFIAIPGQVT EQQQLQTRAN VTRLCQVTST DHGRTWSSPR DLTDAAIGPA YREWSTFAVG
              170 180 190 200 210 220 230 240
PGHCLQLHDR ARSLVVPAYA YRKLHPIQRP IPSAFCFLSH DHGRTWARGH FVAQDTLECQ VAEVETGEQR VVTLNARSHL
              250 260 270 280 290 300 310 320
RARVQAQSTN DGLDFQESQL VKKLVEPPPQ GCQGSVISFP SPRSGPGSPA QWLLYTHPTH SWQRADLGAY LNPRPPAPEA
              330 340 350 360 370 380
WSEPVLLAKG SCAYSDLQSM GTGPDGSPLF GCLYEANDYE EVIFLMFTLK QAFPAEYLPQ
SEQ ID NO: 5
                10 20 30 40 50 60 70 80
MEEVTTCSFN SPLFRQEDDR GITYRIPALL YIPPTHTFLA FAEKRSTRRD EDALHLVLRR GLRIGQLVQW GPLKPLMEAT
                90 100 110 120 130 140 150 160
LPGHRTMNPC PVWEQKSGCV FLFFICVRGH VTERQQIVSG RNAARLCFIY SQDAGCSWSE VRDLTEEVIG SELKHWATFA
              170 180 190 200 210 220 230 240
VGPGHGIQLQ SGRLVIPAYT YYIPSWFFCF QLPCKTRPHS LMIYSDDLGV TWHHGRLIRP MVTVECEVAE VTGRAGHPVL
              250 260 270 280 290 300 310 320
YCSARTPNRC RAEALSTDHG EGFQRLALSR QLCEPPHGCQ GSVVSFRPLE IPHRCQDSSS KDAPTIQQSS PGSSLRLEEE
              330 340 350 360 370 380 390 400
AGTPSESWLL YSHPTSRKQR VDLGIYLNQT PLEAACWSRP WILHCGPCGY SDLAALEEEG LFGCLFECGT KQECEQIAFR
              410 420
LFTHREILSH LQGDCTSPGR NPSQFKSN
SEQ ID NO: 6
                10 20 30 40 50 60 70 80
MGVPRTPSRT VLFERERTGL TYRVPSLLPV PPGPTLLAFV EQRLSPDDSH AHRLVLRRGT LAGGSVRWGA LHVLGTAALA
                90 100 110 120 130 140 150 160
EHRSMNPCPV HDAGTGTVFL FFIAVLGHTP EAVQIATGRN AARLCCVASR DAGLSWGSAR DLTEEAIGGA VQDWATFAVG
              170 180 190 200 210 220 230 240
PGHGVQLPSG RLLVPAYTYR VDRRECFGKI CRTSPHSFAF YSDDHGRTWR CGGLVPNLRS GECQLAAVDG GQAGSFLYCN
              250 260 270 280 290 300 310 320
ARSPLGSRVQ ALSTDEGTSF LPAERVASLP ETAWGCQGSI VGFPAPAPNR PRDDSWSVGP GSPLQPPLLG PGVHEPPEEA
              330 340 350 360 370 380 390 400
AVDPRGGQVP GGPFSRLQPR GDGPRQPGPR PGVSGDVGSW TLALPMPFAA PPQSPTWLLY SHPVGRRARL HMGIRLSQSP
              410 420 430 440 450 460 470 480
LDPRSWTEPW VIYEGPSGYS DLASIGPAPE GGLVFACLYE SGARTSYDEI SFCTFSLREV LENVPASPKP PNLGDKPRGC
CPWS
SEQ ID NO: 7
                10 20 30 40 50 60 70 80
MMSSAAFPRW LSMGVPRTPS RTVLFERERT GLTYRVPSLL PVPPGPTLLA FVEQRLSPDD SHAHRLVLRR GTLAGGSVRW
                90 100 110 120 130 140 150 160
GALHVLGTAA LAEHRSMNPC PVHDAGTGTV FLFFIAVLGH TPEAVQIATG RNAARLCCVA SRDAGLSWGS ARDLTEEAIG
              170 180 190 200 210 220 230 240
GAVQDWATFA VGPGHGVQLP SGRLLVPAYT YRVDRRECFG KICRTSPHSF AFYSDDHGRT WRCGGLVPNL RSGECQLAAV
              250 260 270 280 290 300 310 320
DGGQAGSFLY CNARSPLGSR VQALSTDEGT SFLPAERVAS LPETAWGCQG SIVGFPAPAP NRPRDDSWSV GPGSPLQPPL
              330 340 350 360 370 380 390 400
LGPGVHEPPE EAAVDPRGGQ VPGGPFSRLQ PRGDGPRQPG PRPGVSGDVG SWTLALPMPF AAPPQSPTWL LYSHPVGRRA
              410 420 430 440 450 460 470 480
RLHMGIRLSQ SPLDPRSWTE PWVIYEGPSG YSDLASIGPA PEGGLVFACL YESGARTSYD EISFCTFSLR EVLENVPASP
              490
KPPNLGDKPR GCCWPS
SEQ ID NO: 8
                10 20 30 40 50 60 70 80
MMSSAAFPRW LQSMGVPRTP SRTVLFERER TGLTYRVPSL LPVPPGPTLL AFVEQRLSPD DSHAHRLVLR RGTLAGGSVR
                90 100 110 120 130 140 150 160
WGALHVLGTA ALAEHRSMNP CPVHDAGTGT VFLFFIAVLG HTPEAVQIAT GRNAARLCCV ASRDAGLSWG SARDLTEEAI
              170 180 190 200 210 220 230 240
GGAVQSWATF AVGPGHGVQL PSGRLLVPAY TYRVDRRECF GKICRTSPHS FAFYSDDHGR TWRCGGLVPN LRSGECQLAA
              250 260 270 280 290 300 310 320
VDGGQAGSFL YCNARSPLGS RVQALSTDEG TSFLPAERVA SLPETAWGCQ GSIVGFPAPA PNRPRDDSWS VGPGSPLQPP
              330 340 350 360 370 380 390 400
LLGPGVHEPP EEAAVDPRGG QVPGGPFSRL QPRGDGPRQP GPRPGVSGDV GSWTLALPMP FAAPPQSPTW LLYSHPVGRR
              410 420 430 440 450 460 470 480
ARLHMGIRLS QSPLDPRSWT EPWVIYEGPS GYSDLASIGP APEGGLVFAC LYESGARTSY DEISFCTFSL REVLENVPAS
              490
PKPPNLGDKP RGCCWPS
SEQ ID NO: 9
                10 20 30 40 50 60 70 80
MTSHSPFSRR RLPALLGSLP LAATGLIAAA PPAHAVPTSD GLADVTITQV NAPADGLYSV GDVMTFNITL TNTSGEAHSY
                90 100 110 120 130 140 150 160
APASTNLSGN VSKCRWRNVP AGTTKTDCTG LATHTVTAED LKAGGFTPQI AYEVKAVEYA GKALSTPETI KGATSPVKAN
              170 180 190 200 210 220 230 240
SLRVESITPS SSQENYKLGD TVSYTVRVRS VSDKTINVAA TESSFDDLGR QCHWGGLKPG KGAVYNCKPL THTITQADVD
              250 260 270 280 290 300 310 320
AGRWTPSITL TATGTDGATL QTLTATGNPI NVVGDHPQAT PAPAPDASTE LPASMSQAQH LAANTATDNY RIPAIPPPPM
              330 340 350 360 370 380 390 400
GTCSSPTTSA RRTTATAAAT TPNPNHIVQR RSTDGGKTWS APTYIHQGTE TGKKVYGSDP SYVVDHQTGT IFNFHVKSYD
              410 420 430 440 450 460 470 480
QGWGGSRGGT DPENRGIIQA EVSTSTDNGW TWTHRTITAD ITKDKPWTAR FAASGQGIQI QHGPHAGRLV QQYTIRTAGG
              490 500 510 520 530 540 550 560
PVQAVSVYSD DHGKTWQAGT PIGTGMDENK VVELSDGSLM LNSRASDGSG FRKVAHSTDG GQTWSEPVSD KNLPDSVDNA
              570 580 590 600 610 620 630 640
QIIRAFPNAA PDDPRAKVLL LSHSPNPRPW CRDRGTISMS CDDGASWTTS KVFHEPFVGY TTIAVQSDGS IGLLSEDAHN
              650 660 670 680 690 700 710 720
GADYGGIWYR NFTMNWLGEQ CGQKPAEPSP GRRRRRHPQR HRRRSRPRRP RRALSPRRHR HHPPRPSRAL RPSRAGPGAG
              730 740 750 760 770 780 790 800
AHDRSEHGAH TGSCAQSAPE QTDGPTAAPA PETSSAPAAE PTQAPTVAPS VEPTQAPGAQ PSSAPKPGAT GRAPSVVNPK
              810 820 830 840 850 860 870 880
ATGAATEPGT PSSSASPAPS RNAAPTPKPG MEPDEIDRPS DGTMAQPTGA PARRVPRRRR RRRPAAGCLA RDQRAADPGP
              890 900 910
CGCRGCRRVP AAAGSPFEEL NTRRAGHPAL STD
SEQ ID NO: 10
                10 20 30 40 50 60 70 80
MTTTKSSALR RLSALAGSLA LAVTGIIAAA PPAHATPTSD GLADVTITQT HAPADGIYAV GDVMTFDITL TNTSGQARSF
                90 100 110 120 130 140 150 160
APASTNLSGN VLKCRWSNVA AGATKTDCTG LATHTVTAED LKAGGFTPQI AYEVKAVGYK GEALNKPEPV TGPTSQIKPA
              170 180 190 200 210 220 230 240
SLKVESFTLA SPKETYTVGD VVSYTVRIRS LSDQTINVAA TDSSFDDLAR QCHWGNLKPG QGAVYNCKPL THTITQADAD
              250 260 270 280 290 300 310 320
HGTWTPSITL AATGTDGAAL QTLAATGEPL SVVVERPKAD PAPAPDASTE LPASMSDAQH LAENTATDNY RIPAITTAPN
              330 340 350 360 370 380 390 400
GDLLVSYDER PRDNGNNGGD SPNPNHIVQR RSTDGGKTWS APSYIHQGVE TGRKVGYSDP SYVVDNQTGT IFNFHVKSFD
              410 420 430 440 450 460 470 480
QGWGHSQAGT DPEDRSVIQA EVSTSTDNGW SWTHRTITAD ITRDNPWTAR FAASGQGIQI HQGPHAGRLV QQYTIRTADG
              490 500 510 520 530 540 550 560
VVQAVSVYSD DHGQTWQAGT PTGTGMDENK VVELSDGSLM LNSRASDGTG FRKVATSTDG GQTWSEPVPD KNLPDSVDNA
              570 580 590 600 610 620 630 640
QIIRPFPNAA PSDPRAKVLL LSHSPNPRPW SRDRGTISMS CDNGASWVTG RVFNEKFVGY TTIAVQSDGS IGLLSEDGNY
              650 660 670 680 690 700 710 720
GGIWYRNFTM GWVGDQCSQP RPEPSPSPTP SAAPSAEPTS EPTTAPAPEP TTAPSSEPSV SPEPSSSAIP APSQSSSATS
              730 740 750 760 770 780 790
GPSTEPDEID RPSDGAMAQP TGGAGRPSTS VTGATSRNGL SRTGTNALLV LGVAAAAAAG GYLVLRIRRA RTE
SEQ ID NO: 11
                10 20 30 40 50 60 70 80
MNYKSLDRKQ RYGIRKFAVG AASVVIGTVV FGANPVLAQE QANAAGNATE TVEPGQGLSE LPKEASSGDL AHLDKDLAGK
                90 100 110 120 130 140 150 160
LAAAQDNGVE VDQDHLKKNE SAESETPSST ETPAEEANKE EESEDQGAIP RDYYSRDLKN ANPVLEKEDV ETNAANGQRV
              170 180 190 200 210 220 230 240
DLSNELDKLK QLKNATVHME FKPDASAPRF YNLFSVSSDT KENEYFTMSV LDNTALIEGR GANGEQFYDK YTDAPLKVRP
              250 260 270 280 290 300 310 320
GQWNSVTFTV EQPTTELPHG RVRLYVNGVL SRTSLKSGNF IKDMPDVNQA QLGATKRGNK TVWASNLQVR NLTVYDRALS
              330 340 350 360 370 380 390 400
PDEVQTRSQL FERGELEQKL PEGAKVTEKE DVFEGGRNNQ PNKDGIDSYR IPALLKTDKG TLIAGTDERR LHHSDWGDIG
              410 420 430 440 450 460 470 480
MVVRRSSDNG KTWGDRIVIS NPRDNEHAKH ADWPSPVNID MALVQDPETK RIFAIYDMFL ESKAVFSLPG QAPKAYEQVG
              490 500 510 520 530 540 550 560
DKVYQVLYKQ GESGRYTIRE NGEVFDPQNR KTDYRVVVDP KKPAYSDKGD LYKGNELIGN IYFEYSEKNI FRVSNTNYLW
              570 580 590 600 610 620 630 640
MSYSDDDGKT WSAPKDITHG IRKDWMHFLG TGPGTGIALR TGPHKGRLVI PVYTTNNVSY LSGSQSSRVI YSDDHGETWQ
              650 660 670 680 690 700 710 720
AGEAVNDNRP VGNQTIHSST MNNPGAQNTE STVVQLNNGD LKLFMRGLTG DLQVATSHDG GATWDKEIKR YPQVKDVYVQ
              730 740 750 760 770 780 790 800
MSAIHTMHEG KEYILLSNAG GPGRNNGLVH LARVEENGEL GKFAYNSLQE LGNGEYGLLY EHADGNQNDY EHADGNQNDY
              810 820 830 840 850 860 870 880
TLSYKKFNWD FLSRDRISPK EAKVKYAIQK WPGIIAMEFD SEVLVNKAPT LQLANGKTAT FMTQYDTKTL LFTIDPEDMG
              890 900 910 920 930 940 950 960
QRITGLAEGA IESMHNLPVS LAGSKLSDGI NGSEAAIHEV PEFTGGVNAE EAAVAEIPEY TGPLATVGEE VAPTVEKPEF
              970 980 990 1000 1010 1020 1030 1040
TGGVNAEEAP VAEMPEYTGP LSTVGEEVAP TVEKPEFTGG VNAVEAAVHE LPEFKGGVNA VLAASNELPE YRGGANFVLA
            1050 1060 1070 1080 1090 1100 1110 1120
ASNDLPEYIG GVNGAEAAVH ELPEYKGDTN LVLAAADNKL SLGQDVTYQA PAAKQAGLPN TGSKETHSLI SLGLAGVLLS
            1130
LFAFGKKRKE
SEQ ID NO: 12
                10 20 30 40 50 60 70 80
MSDLKKYEGV IPAFYACYDD QGEVSPERTR ALVQYFIDKG VQGLYVNGSS GECIYQSVED RKLILEEVMA VAKGKLTIIA
                90 100 110 120 130 140 150 160
HVACNNTKDS MELARHAESL GVDAIATIPP IYFRLPEYSV AKYWNDISAA APNTDYVIYN IPQLAGVALT PSLYTEMLKN
              170 180 190 200 210 220 230 240
PRVIGVKNSS MPVQDIQTFV SLGGEDHIVF NGPDEQFLGG RLMGAKAGIG GTYGAMPELF LKLNQLIAEK DLETARELQY
              250 260 270 280 290 300
AINAIIGKLT SAHGNMYGVI KEVLKINEGL NIGSVRSPLT PVTEEDRPVV EAAAQLIRET KERFL
SEQ ID NO: 13
                10 20 30 40 50 60 70 80
MNQRHFDRKQ RYGIRKFTVG AASVVIGAVV FGVAPALAQE APSTNGETAG QSLPELPKEV ETGNLTNLDK ELADKLSTAT
                90 100 110 120 130 140 150 160
DKGTEVNREE LQANPGSEKA AETEASNETP ATESEDEKED GNIPRDFYAR ELENVNTVVE KEDVETNPSN GQRVDMKEEL
              170 180 190 200 210 220 230 240
DKLKKLQNAT IHMEFKPDAS APRFYNLFSV SSDTKVNEYF TMAILDNTAI VEGRDANGNQ FYGDYKTAPL KIKPGEWNSV
              250 260 270 280 290 300 310 320
TFTVERPNAD QPKGQVRVYV NGVLSRTSPQ SGRFIKDMPD VNQVQIGTTK RTGKNFWGSN LKVRNLTVYD RALSPEEVKK
              330 340 350 360 370 380 390 400
RSQLFERGEL EKKLPEGAKV TDKLDVFQGG ENRKPNKDGI ASYRIPALLK TDKGTLIAGA DERRLHHSDW GDIGMVVRRS
              410 420 430 440 450 460 470 480
DDKGKTWGDR IVISNPRDNE NARRAHAGSP VNIDMALVQD PKTKRIFSIF DMFVEGEAVR DLPGAKPQAY EQIGNKVYQV
              490 500 510 520 530 540 550 560
KYKKGEAGHY TIRENGEVFD PENRKTEYRV VVDPKKPAYS DKGDLYKGEE LIGNVYFDYS DKNIFRVSNT NYLWMSYSDD
              570 580 590 600 610 620 630 640
DGKTWSAPKD ITYGIRKDWM HFLGTGPGTG IALHSGPHKG RLVIPAYTTN NVSYLGGSQS SRVIYSDDHG ETWHAGEAVN
              650 660 670 680 690 700 710 720
DNRPIGNQTI HSSTMNNPGA QNTESTVVQL NNGDLKLFMR GLTGDLQVAT SKDGGATWEK DVKRYADVKD VYVQMSAIHT
              730 740 750 760 770 780 790 800
VQEGKEYIIL SNAGGPGRYN GLVHVARVEA NGDLTWIKHN PIQSGKFAYN SLQDLGNGEF GLLYEHATAT QNEYTLSYKK
              810 820 830 840 850 860 870 880
FNWDFLSKDG VAPTKATVKN AVEMSKNVIA LEFDSEVLVN QPPVLKLANG NFATFLTQYD SKTLLFAASK EDIGQEITEI
              890 900 910 920 930 940 950 960
IDGAIESMHN LPVSLEGAGV PGGKNGAKAA IHEVPEFTGA VNGEGTVHED PAFEGGINGE EAAVHDVPDF SGGVNGEVAA
              970 980 990 1000 1010 1020 1030 1040
IHEVPEFTGG INGEEAAKLE LPSYEGGANA VEAAKSELPS YEGGANAVEA AKLELPSYES GAHEVQPASS LNPTLADSVN
            1050 1060 1070 1080 1090 1100 1110
SEQ ID NO: 14
                10 20 30 40 50 60 70 80
MNQSSLNRKN RYGIRKFTIG VASVAIGSVL FGITPALAQE TTTNIDVSKV ETSLESGAPV SEPVTEVVSG DLNHLDKDLA
                90 100 110 120 130 140 150 160
DKLALATNQG VDVNKHNLKE ETSKPEGNSE HLPVESNTGS EESIEHHPAK IEGADDAVVP PRDFFARELT NVKTVFERED
              170 180 190 200 210 220 230 240
LATNTGNGQR VDLAEELDQL KQLQNATIHN EFKPDANAPQ FYNLFSVSSD KKKDEYFSMS VNKGTAMVEA RGADGSHFYG
              250 260 270 280 290 300 310 320
SYSDAPLKIK PGQWNSVTFT VERPKADQPN GQVRLYVNGV LSRTNTKSGR FIKDMPDVNK VQIGATRRAN QTMWGSNLQI
              330 340 350 360 370 380 390 400
RNLTVYNRAL TIEEVKKRSH LFERNDLEKK LPEGAEVTEK KDIFESGRNN QPNGEGINSY RIPALLKTDK GTLIAGGDER
              410 420 430 440 450 460 470 480
RLHHFDYGDI GMVIRRSQDN GKTWGDKLTI SNLRDNPEAT DKTATSPLNI DMVLVQDPTT KRIFSIYDMF PETRAVFGMP
              490 500 510 520 530 540 550 560
NQPEKAYEEI GDKTYQVLYK QGETERYTLRDNGEIFNSQN KKTEYRVVVN PTEAGFRDKG DLYKNQEILIG NIYFKQSDKN
              570 580 590 600 610 620 630 640
PFRVANTSYL WMSYSDDDGK TWSAPKDITP GIRQDWMKFL GTGPGTGIVL RTGAHKGRIL VPAYTTNNIS HLGGSQSSRL
              650 660 670 680 690 700 710 720
IYSDDHGQTW HAGESPNDNR PVGNSVIHSSNMNKSSAQNT IESTVLQLNNG DVKLFMRGLT GDLQVATSKD GGVTWEKTIK
              730 740 750 760 770 780 790 800
RYPEVKDAYV QMSAIHTMHD GKEYILLSNA AGPGRERKNG LVHLARVEEN GELTWLHNNP IQNGEFAYNS LQELGGGEYG
              810 820 830 840 850 860 870 880
LLYEHRENGQ NYYTLSYKKF NWDFVSDKLI SPTEAKVSQA YEMGKGVFGL EFDSEVLVNR APILRLANGR TAVFMTQYDS
              890 900 910 920 930 940 950 960
KTLLFAVDKK DIGQEITGIV DGSIESMHNL TVNLAGAGIP GGMNAAESVE HYTEEYTGVL GTSGVEGVPT ISVPEYEGGV
              970 980 990 1000 1010 1020 1030 1040
NSELALVSEK EDYRGGVNSA SSVVTEVLEY TGPLSTVGSE DAPTVSVLEY EGGVNIDSPE VTEAPEYKEP IGTSGYELAP
            1050 1060 1070 1080 1090 1100 1110 1120
TVDKPAYTGT IEPLEKEENS GAIIEEGNVS YITENNNKPL ENNNVTTSSI ISESSKLKHT LKNATGSVQI HASEEVLKNV
            1130 1140 1150 1160 1170 1180 1190 1200
KDVKIQEVKV SSLSSLNYKA YDIQLNDASG KAVQPKGTVI VTFAAEQSVE NVYYVDSKGN LHTLEFLQKD GEVTFETNHF
            1210 1220 1230 1240 1250 1260 1270 1280
SIYAMTFQLS LDNVVLDNHR EDKNGEVNSA SPKLLSINGH SQSSQLENKV SNNEQSKLPN TGEDKSISTV LLGFVGVILG
            1290
AMIFYRRKDS EG
SEQ ID NO: 15
                10 20 30 40 50 60 70 80
MDKKKIILTS LASVAVLGAA LAASQPSLVK AEEQPTASQP AGETGTKSEV TSPEIKQAEA DAKAAEAKVT EAQAKVDTTT
                90 100 110 120 130 140 150 160
PVADEAAKKL ETEKKEADEA DAAKTKAEEA KKTADDELAA AKEKAAEADA KAKEEAKKEE DAKKEEADSK EALTEALKQL
              170 180 190 200 210 220 230 240
PDNELLDKKA KEDLLKAVEA GDLKASDILA ELADDDKKAE ANKETEKKLR NKDQANEANV ATTPAEEAKS KDQLPADIKA
              250 260 270 280 290 300 310 320
GIDKAEKADA ARPASEKLQD KADDLGENVD ELKKEADALK AEEDKKAETL KKQEDTLXEA KEALKSAKDN GFGEDITAPL
              330 340 350 360 370 380 390 400
EKAVTAIEKE RDAAQNAFDQ AASDTKAVAD ELNKLTDEYN KTLEEVKAAK EKEANEPAKP VEEEPAKPAE KTEAEKAAEA
              410 420 430 440 450 460 470 480
KTEADAKVAE LQKKADEAKT KADEATAKAT KEAEDVKAAE KAKEEADKAK TDAEAELAKA KEEAEKAKAK VEELKKEEKD
              490 500 510 520 530 540 550 560
NLEALKAALD QLEKDIDADA TITNKEEAKK ALGKEDILAA VEKGDLTAGD VLKELENQNA TAEATKDQDP QADEIGATKQ
              570 580 590 600 610 620 630 640
EGKPLSELPA ADKEKLDAAY NKEASKPIVK KLQDIADDLV EKIEKLTKVA DKDKADATEK AKAVEEKNAA LKKQKETLDK
              650 660 670 680 690 700 710 720
AKAALETAKK NQADQAIDQG LQDAVTKLEA SFASAKTAAD EAQAKFDEVN EVVKAYKAAI DELTDDYNAT LGHIENLKEV
              730 740 750 760 770 780 790 800
PKGEEPKDFS GGVNDDEAPS STPNTNEFTG GANADAPATA PNANEFAGGV NDEEAPTTEN KEPFNGGVND EEAPTVPNKP
              810 820 830 840 850 860 870 880
EGEAPKPTGE NAKDAPVVKL PEFGANNPEI KKILDEIAKV KEQIKDGEEN GSEDYYVEGL KERLADLEEA FDTLSKNLPA
              890 900 910 920 930 940 950 960
VNKVPEYTGP VTPENGQTQP AVNTPGGQQG GSSQQTPAVQ QGGSGQQAPA VQQGGSNQQV PAVQQTNTPA VAGTSQDNTY
              970 980 990 1000
QAPAAKEEDK KELPNTGGQE SAALASVGFL GLLLGALPFV
SEQ ID NO: 16
                10 20 30 40 50 60 70 80
MKYRDFDRKR RYGIRKFAVG AASVVIGTVV FGANPVLAQE QANAAGANTE TVEPGQGSLE LPKEASSGDL AHLDKDLAGK
                90 100 110 120 130 140 150 160
LAAAQDNGVE VDQDHLKKNE SAESETPSST ETPAEGTNKE EESEDQGAIP RDYYSRDLKN ANPVLEKEDV ETNAANGQRV
              170 180 190 200 210 220 230 240
DLSNELDKLK QLKNATVHME FKPDASAPRF YNLFSVSSDT KENEYFTISV LDNTALIEGR GANGEQFYDK YTDAPLKVRP
              250 260 270 280 290 300 310 320
GQWNSVFTFV EQPTTELPHG RVRLYVNGVL SRTSLKSGNF IKDMPDVNQA QLGATKRGNK TVWASNLQVR NLTVYDRALS
              330 340 350 360 370 380 390 400
PDEVQTRSQL FERGELEQKL PEGAKVTEKE DVFEGGRNNQ PNKDGIKSRY IPALLKTDKG TLIAGTDERR LHHSDWGDID
              410 420 430 440 450 460 470 480
MVVRRSSDNG KTWGDRIVIS NPRDNEHAKH ADWPSPVNID MALVQDPETK RIFAIYDMFL ESKAVFSLPG QAPKAYEQVG
              490 500 510 520 530 540 550 560
DKVYQVLYKQ GESGRYTIRE NGEVFDPQNR KTDYRVVVDP KKPAYDDKGD LYKGNELIGN IYFEYSEKNI FRVSNTNYLW
              570 580 590 600 610 620 630 640
MSYSDDDGKT WSAPKDITHG IRKDWMHFLG TGPGTGIALR TGPHKGRLVI PVYTTNNVSY LSGSQSSRVI YSDDHGETWQ
              650 660 670 680 690 700 710 720
AGEAVNDNRP VGNQTIHSST NMMPGAQNTE STVVQLNNGD LKLFMRGLTG DLQVATSHDG GATWDKEIKR YPQVKDVYVQ
              730 740 750 760 770 780 790 800
MSAIHTMHEG KEYILLSNAG GPGRNNGLVH LARVEENGEL TWLKHNPIQS GKFAYNSLQD LGNGEYGLLY EHADGNQNDY
              810 820 830 840 850 860 870 880
TLSYKKFNWD FLTKDWISPK EAKVKYAIEK WPGILAMEFD SEVLVNKAPT LQLANGKTAR FMTQYDTKTL LFTVDSEDMG
              890 900 910 920 930 940 950 960
QKVTGLAEGA IESMHNLPVS VAGTKLSNGM NGSEAAVHEV PEYTGPLGTA GEEPAPTVEK PEFTGGVNGE EAAVHEVPEY
              970 980 990 1000 1010 1020 1030 1040
TGPLGTSGEE PAPTVEKPEF TGGVNAVEAA AHEVPEYTGP LGTSGKEPAP TVEKPEYTGG VNAVEAAVHE VEPYTGPLAT
            1050 1060 1070 1080 1090 1100 1110 1120
VGEEAAPKVD KPEFTGGVNA VEAAVHELPE YTGGVNAADA AVHEIAEYKG ADSLVTLAAE DYTYKAPLAQ QTLPDTGNKE
            1130 1140
SSLLASLGLT AFFLGLFAMG KKREK
SEQ ID NO: 17
                10 20 30 40 50 60 70 80
MEKIWREKSC RYSIRKLTVG TASVLLGAVF LASHTVSADT IKVKQNESTL EKTTAKTDTV TKTTESTEHT QPSEAIDHSK
                90 100 110 120 130 140 150 160
QVLANNSSSE SKPTEAKVAS ATTNQASTEA IVKPNENKET EKQELPVTEQ SNYQLNYDRP TAPSYDGWEK QALPVGNGEM
              170 180 190 200 210 220 230 240
GAKVFGLIGE ERIQYNEKTL WSGGPRPDST DYNGGNYRER YKILAEIRKA LEDGDRQKAK RLAEQNLVGP NNAQYGRYLA
              250 260 270 280 290 300 310 320
FGDIFMVFNN QKKGLDTVTD YHRGLDITEA TTTTSYTQDG TTFKRETFSS YPDDVTVTHL TQKGDKKLDF TVWNSLTEDL
              330 340 350 360 370 380 390 400
LANGDYSAEY SNYKSGHVTT DPNGILLKGT VKDNGLQFAS YLGIKTDGKV TVHEDSLTIT GASYATLLLS AKTNFAQNPK
              410 420 430 440 450 460 470 480
TNYRKDIDLE KTVKGIVEAA QGKYYETLKR NHIKDYQSLF NRVKLNLGGS NIAQTTKEAL QTYNPTKGQK LEELFFQYGR
              490 500 510 520 530 540 550 560
YLLISSSRSR TDALPANLQG VWNAVDNPPW NADYHLNVNL QMNYWPAYMS NLAETAKPMI NYIDDMRYYG RIAAKEYAGI
              570 580 590 600 610 620 630 640
ESKDGQENGW LVHTQATPFG WTTPGWNYYW GWSPAANAWM MQNVYDYYKF TKDETYLKEK IYPMLKETAK FWNSFLHYDQ
              650 660 670 680 690 700 710 720
ASDRWVSSPS YSPEHGTITI GNTFDQSLVW QLFHDYMEVA NHLNVDKDLV TEVKAKFDKL KPLHINKEGT IKEWYEEDSP
              730 740 750 760 770 780 790 800
QFTNEGIENN HRHVSHLVGL FPGTLFSKDQ AEYLEAARAT LNHRGDGGTG WSKANKINLW ARLLDGNRAH RLLAEQLKYS
              810 820 830 840 850 860 870 880
TLENLWDTHA PFQIDGNFGA TSGIAEMLLQ SHTGYIAPLP ALPDAWKDGQ VSGLVARGNF EVSMQWKDKN LQSLSFLSNV
              890 900 910 920 930 940 950 960
GGDLVVDYPN IEASQVKVNG KPVKATVLKD GRIQLATQKG DVITFEHFSG RVTSLTAVRQ NGVTAELTFN QVEGATHYVI
              970 980 990 1000 1010 1020 1030 1040
QRQVKDESGQ TSATREFVTN QTHFIDRSLD PQLAYTYTVK AMLGNVSTQV SEKANVETYN QLMDDRDSRI QYGSAFGNWA
            1050 1060 1070 1080 1090 1100 1110 1120
DSELFGGTEK FADLSLGNYT DKDATATIPF NGVGIEYIGL KSSQLGIAEV KIDGKSVGEL DFYTAGATEK GSLIGRFTGL
            1130 1140 1150 1160 1170 1180 1190 1200
SDGAHVMTIT VKQEHKHRGS ERSKISLDYF KVLPGQGTTI EKMDDRDSRI QYGSQFKDWS DTELYKSTEK YADINNSDPS
            1210 1220 1230 1240 1250 1260 1270 1280
TASEAQATIP FTGTGIRIYG LKTSALGKAL VTLDGKEMPS LDFYTAGATQ KATLIGEFTN LTDGNHILTL KVDPNSPAGR
            1290 1300 1310 1320 1330 1340 1350 1360
KKISLDSFDV IKSPAVSLDS PSIAPLKKGD KNISLTLPAG DWEAIAVTFP GIKDPLVLRR IDDNHLVTTG DQTVLSIQDN
            1370 1380 1390 1400 1410 1420 1430 1440
QVQIPIPDET NRKIGNAIEA YSIQGNTTSS PVVAVFTKKD EKKVENQQPT TSKGDDPAPI VEIPEYTKPI GTAGLEQPPT
            1450 1460 1470 1480 1490 1500 1510 1520
VSIPEYTQPI GTAGLEQAPT VSIPEYTKPV GTAGIEQAPT VSIPEYTKPI GTAGLEQAPT VSIPEYTQPI GTAGLEQPPT
            1530 1540 1550 1560 1570 1580 1590 1600
VSIPEYTKSI GTAGLEQPPV VNVPEYTQPI GTAGIEQPPT VSIPEYTKPI GTAGQEQALT VSIPEYTKPI GTAGQEQAPT
            1610 1620 1630 1640 1650 1660 1670 1680
VSVPEYKLRV LKDERTGVEI IGGATDLEGI SHISSRRVLA QELFGKTYDA YDLHLKNSTD QSLQPKGSVL VRLPISSAVE
            1690 1700 1710 1720 1730 1740 1750 1760
NVYYLTPSKE LQALDFTIRE GMAEFTTSHF STYAVVYQAN GASTTAEQKP SETDIKPLAN SSEQVSSSPD LVQSTNDSPK
            1770 1780 1790
EQLPATGETS NPLLFLSGLS LVLTATFLLK SKKDESN
SEQ ID NO: 18
                10 20 30 40 50 60 70 80
MKQYFLEKGR IFSIRKLTVG VASVAVGLTF FASGNVAASE LVTEPKLEVD GQSKEVADVK HEKEEAVKEE AVKEEVTEKT
                90 100 110 120 130 140 150 160
ELTAEKATEE AKTAEVAGDV LPEEIPDRAY PDTPVKKVDT AAIVSEQESP QVETKSILKP TAVAPTEGEK ENRAVINGGQ
              170 180 190 200 210 220 230 240
DLKRINYEGQ PATSAAMVYT IFSSPLAGGG SRRYLNSGSG IFVAPNIMLT VAHNFLVKDA DTNAGSIRGG DTTKFYYNVG
              250 260 270 280 290 300 310 320
SNTAKNNSLP TSGNTVLFKE KDIHFWNKEK FGEGIKNDLA LVVAPVPLSI ASPNKAATFT PLAEHREYKA GEPVSTIGYP
              330 340 350 360 370 380 390 400
TDSTSPELKE PIVPGQLYKA DGVVKGTEKL DDKGAVGITY RLTSVSGLSG GGIINGDGKV IGIHQHGTVD NMNIAEKDRF
              410 420 430 440 450 460 470 480
GGGLVLSPEQ LAWVKEIIDK YGVKGWYQGD NGNRYYFTPE GEMIRNKTAV IGKNKYSFDQ NGIATLLEGV DYGRVVVEHL
              490 500 510 520 530 540 550 560
DQKDNPVKEN DTFVEKTEVG TQFDYNYKTE IEKTDFYKKN KEKYEIVSID GKAVNKQLKD TWGEDYSVVS KAPAGTRVIK
              570 580 590 600 610 620 630 640
VVYKVNKGSF DLRYRLKGTD QELAPATVDN NDGKEYEVSF VHRFQAKEIT GYRAVNASQE ATIQHKGVNQ VIFEYEKIED
              650 660 670 680 690 700 710 720
PKPATPATPV VDPKDEETEI GNYGPLPSKA QLDYHKEELA AFIHYGMNTY TNSEWGNGRE NPQNFNPTNL DTDQWIKTLK
              730 740 750 760 770 780 790 800
DAGFKRTIMV VKHHDGFVIY PSQYTKHTVA ASPWKDGKGD LLEEISKSAT KYDMNMGVYL SPWDANNPKY HVSTEKEYNE
              810 820 830 840 850 860 870 880
YYLNQLEKIL GNPKYGNKGK FIEVWMDGAR GSGAQKVTYT FDEWFKYIKK AEGDIAIFSA QPTSVRWIGN ERGIAGDPVW
              890 900 910 920 930 940 950 960
HKVKKAKITD DVKNEYLNHG DPEGDMYSVG EADVSIRSGW FYHDNQQPKS IKDLMDIYFK SVGRGTPLLL NIPPNKEGKF
              970 980 990 1000 1010 1020 1030 1040
ADADVARLKE FRATLDQMYA TDFAKGATVT ASSTRKNHLY QASNLTDGKD DTSWALSNDA KTGEFTVDLG QKRRFDVVEL
            1050 1060 1070 1080 1090 1100 1110 1120
KEDIAKGQRI SGFKVEVELN GRWVPYGEGS TVGYRRLVQG QPVEAQKIRV TITNSQATPI LTNFSVYKTP SSIEKTDGYP
            1130 1140 1150 1160 1170 1180 1190 1200
LGLDYHSNTT ADKANTTWYD ESEGIRGTSM WTNKKDASVT YRFNGTKAYV VSTVDPNHGE MSVYVDGQKV ADVQTNNAAR
            1210 1220 1230 1240 1250 1260 1270 1280
KRSQMVYETD DLAPGEHTIK LVNKTGKAIA TEGIYTLNNA GKGMFELKET TYEVQKGQPV TVTIKRVGGS KGAATVHVVT
            1290 1300 1310 1320 1330 1340 1350 1360
EPGTGVHGKV YKDTTADLTF QDGETEKTLT IPTIDFTEQA DSIFDFKVKM TSASDNALLG FASEATVRVM KADLLQKDQV
            1370 1380 1390 1400 1410 1420 1430 1440
SHDDQASQLD YSPGWHHETN SAGKYQNTES WASFGRLNEE QKKNASVTAY FYGTGLEIKG FVDPGHGIYK VTLDGKELEY
            1450 1460 1470 1480 1490 1500 1510 1520
QDGQGNATDV NGKKYFSGTA TTRQGDQTLV RLTGLEEGWH AVTLQLDPRK NDTSRNIGIQ VDKFITRGED SALYTKEELV
            1530 1540 1550 1560 1570 1580 1590 1600
QAMKNWKDEL AKFDQTSLKN TEPARQAFKS NLDKLSEQLS ASPANAQEIL KIATALQAIL DKEENYGKED TPTSEQPEEP
            1610 1620 1630 1640 1650 1660 1670 1680
NYDKAMASLS EAIQNKSKEL SSDKEAKKKL VELSEQALTA IQEAKTQDAV DKALQAALTS INQLQATPKE EVKPSQPEEP
            1690 1700 1710 1720 1730 1740 1750 1760
NYDKAMASLA EAIQNKSKEL GSDKESKKKL VELSEQALTA IQEAKTQDAV DKALQAALTS INQLQATPKE EAKPSQPEEP
            1770 1780 1790 1800 1810 1820 1830 1840
NYDKAMASLA EAIQNKSKEL GSDKEAKKKL VELSEQALTA IQEAKTQDAV DKALQAALTS INQLQATPKE EVKHSIVPTD
            1850 1860 1870 1880 1890 1900 1910 1920
GDKELVQPQP SLEVVEKVIN FKKVKQEDSS LPKGETRVTQ VGRAGKERIL TEVAPDGSRT IKLREVVEVA QDEIVLVGTK
            1930 1940 1950 1960 1970 1980 1990 2000
KEESGKIASS VHEVPEFTGG VIDSEATIHN LPEFTGGVTD SEAAIHNLPE FTGGVTDSEA AIHNLPEFTG GMTDSEAAIH
            2010 2020 2030 2040 2050 2060 2070 2080
NLPEFTGGMT DSEGVAHGVS NVEEGVPSGE ATSHQESGFT SDVTDSETTM NEIVYKNDEK SYVVPPMLED KTYQAPANRQ
            2090 2100 2110
EVLPKTGSED GSAFASVGII GMFLGMIGIV KRKKD
SEQ ID NO: 19
                10 20 30 40 50 60 70 80
MSGLKKYEGV IPAFYACYDD AGEVSPERTR ALVQYFIDKG VQGLYVNGSS GECIYQSVED RKLILEEVMA VAKGKLTIIA
                90 100 110 120 130 140 150 160
HVACNNTKDS IELARHAESL GVDAIATIPP IYFRLPEYSV AKYWNDISAA APNTDYVIYN IPQLAGVALT PSLYTEMLKN
              170 180 190 200 210 220 230 240
PRVIGVKNSS MPVQDIQTFV SLGGDDHIVF NGPDEQFLGG RLMGAKAGIG GTYGAMPELF LKLNQLIADK DLETARELQY
              250 260 270 280 290 300
AINAIIGKLT AAHGNMYCVI KEVLKINEGL NIGSVRSPLT PVTEEDRPVV EAAAQILIRES KERFL
SEQ ID NO: 20
                10 20 30 40 50 60 70 80
MANNTLLAKT RRYVCLVVFC CLMAMMHLSG QEVTMWGDSH GVAPNQVRRT LVKVALSESL PPGAKQIRIG FSLPKETEEK
                90 100 110 120 130 140 150 160
VTALYLLVSD SLAVRDLPDY KGRVSYDSFP ISKEDRTTAL SADSVAGRCF FYLAADIGPV ASFSRSDTLT ARVEELAVDG
              170 180 190 200 210 220 230 240
RPLPLKELSP ASRRLYREYE ALFVPGDGGS RNYRIPSILK TANGTLIAMA DRRKYNQTDL PEDIDIVMRR STDGGKSWSD
              250 260 270 280 290 300 310 320
PRIIVQGEGR NHGFGDVALV QTQAGKLLMI FVGGVGLWQS TPDRPQRTYI SESRDEGLTW SPPRDITHIF FGKDCADPGR
              330 340 350 360 370 380 390 400
SRWLASFCAS GQGLVLPSGR VMFVAAIRES GQEYVLNNYV LYSDDEGGTW QLSDCAYHRG DEAKLSLMPD GRVLMSVRNQ
              410 420 430 440 450 460 470 480
GRQESRQRFF ALSSDDGLTW ERAKQFEGIH DPGCNGAMLQ VKRNGRNQML HSLPLGPDGR RDGAVYLFDH VSGRWSAPVV
              490 500 510 520
VNSGSSAYSD MTLLADGTIG YFVEEDDEIS LVFIRFVLDD LFDARQ
SEQ ID NO: 21
                10 20 30 40 50 60 70 80
MTKKSSISRR SFLKSTALAG AAGMVGTGGA ATLLTSCGGG ASSNENANAA NKPLKEPGTY YVPELPDMAA DGKELKAGII
                90 100 110 120 130 140 150 160
GCGGRGSGAA MNFLAAANGV SIVALGDTFQ DRVDSLAQKL KDEKNIDIPA DKRFVGLDAY KQVIDSDVDV VIVATPPNFR
              170 180 190 200 210 220 230 240
PIHFQYAVEK SKHCFLEKPI CVDAVGYRTI MATAKQAQAK NLCVITGTQR HHQRSYIASY QQIMNGIAGE ITGGTVYWNQ
              250 260 270 280 290 300 310 320
SMLWYRERQA GWSDCEWMIR DWVNWKWLSG DHIVEQHVHN IDVFTWFSGL KPVKAVGFGS RQRRITGDQY DNFSIDFTME
              330 340 350 360 370 380 390 400
NGIHLHSMCR QIDGCANNVS EFIQGTKGSW NSTDMGIKDL AGNVIWKYDV EAEKASFKQN DPYTLEHVNW INTIRAGKSI
              410 420 430 440 450 460
DQASETAVSN MAAIMGRESA YTGEETTWEA MTAAALDYTP ADLNLGKMDM KPFVVPVPGK PLEKK
SEQ ID NO: 22
                10 20 30 40 50 60 70 80
MKKFFWIIGL FISMLTTRAA DSVYVQNPQI PILIDRTDNV LFRIRIPDAT KGDVLNRLTI RFGNEDKLSE VKAVRLFYAG
                90 100 110 120 130 140 150 160
TEAGTKGRSR FAPVTYVSSH NIRNTRSANP SYSVRQDEVT TVANTLTLKT RQPMVKGINY FWVSVEMDRN TSLLSKLTPT
              170 180 190 200 210 220 230 240
VTEAVINDKP AVIAGEQAAV RRMGIGVRHA GDDGSASFRI PGLVTTNEGT LLGVYDVRYN NSVDLQEHID VGLSRSTDKG
              250 260 270 280 290 300 310 320
QTWEPMRIAM SFGETDGLPS GQNGVGDPSI LVDERTNTVW VVAAWTHGMG NARAWTNSMP GMTPDETAQL MMVKSTDDGR
              330 340 350 360 370 380 390 400
TWSEPINITS QVKDPSWCFL LQGPGRGITM RDGTLVFPIQ FIDSLRVPHA GIMYSKDRGE TWHISQPART NTTEAQVAEV
              410 420 430 440 450 460 470 480
EPGVLMLNMR DNRGGSRAVS ITRDLGKSWT EHSSNRSALP ESICMASLIS VKAKDNIIGK DLLFFSNPNT TEGRHHITIK
              490 500 510 520 530
ASLDGGVTWL PAHQVLLDEE DGWGYSCLSM IDRETVGIFY ESSVAHMTFQ AVKIKDLIR
SEQ ID NO: 23
                10 20 30 40 50 60 70 80
MTWLLCGRGK WNKVKRMMNS VFKCLMSAVC AVALPAFGQE EKTGFPTDRA VTVFSAGEGN PYASIRIPAL LSIGKGQLLA
                90 100 110 120 130 140 150 160
FAEGRYKNTD QGENDIIMSV SKNGGKTWSR PRAIAKAHGA TFNNPCPVYD AKTRTVTVVF QRYPAGVKER QPNIPDGWDD
              170 180 190 200 210 220 230 240
EKCIRNFMIQ SRNGGSSWTK PQEITKTTKR PSGVDIMASG PNAGTQLKSG AHKGRLVIPM NEGPFGKWVI SCIYSDDGGK
              250 260 270 280 290 300 310 320
SWKLGQPTAN MKGMVNETSI AETDNGGVVM VARHWGAGNC RRIAWSQDGG ETWGQVEDAP ELFCDSTQNS LMTYSLSDQP
              330 340 350 360 370 380 390 400
AYGGKSRILF SGPSAGRRIK GQVAMSYDNG KTWPVKKLLG EGGFAYSSLA MVEPGIVGVL YEENQEHIKK LKFVPITMEW
              410
LTDGEDTGLA PGKKAPVLK
SEQ ID NO: 24
                10 20 30 40 50 60 70 80
MGLGLLCALG LSIPSVLGKE SFEQARRGKF TTLSTKYGLM SCRNGVAEIG GGGKSGEASL RMFGGQDAEL KLDLKDTPSR
                90 100 110 120 130 140 150 160
EVRLSAWAER WTGQAPFEFS IVAIGPNGEK KIYDGKDIRT GGFHTRIEAS VPAGTRSLVF RLTSPENKGM KLDDLFLVPC
              170 180 190 200 210 220 230 240
IPMKVNPQVE MASSAYPVMV RIPCSPVLSL NVRTDGCLNP QFLTAVNLDF TGTTKLSDIE SVAVIRGEEA PIIHHGEEPF
              250 260 270 280 290 300 310 320
PKDSSQVLGT VKLAGSARPQ ISVKGKMELE PGDNYLWACV TMKEGASLDG RVVVRPASVV AGNKPVRVAN AAPVAQRIGV
              330 340 350 360 370 380 390 400
AVVRHGDFKS KFYRIPGLAR SRKGTLLAVY DIRYNHSGDL PANIDVGVSR STDGGRTWSD VKIAIDDSKI SPSLGATRGV
              410 420 430 440 450 460 470 480
GDPAILVDEK TGRIWVAAIW SHRHSIWGSK SGDNSPEACG QLVLAYSDDD GLTWSSPINI TEQTKNKDWR ILFNGPGNGI
              490 500 510 520 530 540 550 560
CMKDGTLVFA AQYWDGKGVP WSTIVYSKDR GKTWHCGTGV NQQTTEAQVI ELEDGSVMIN ARCNWGGSRI VGVTKDLGQT
              570 580 590 600 610 620 630 640
WEKHPTNRTA QLKEPVCQGS LLAVDGVPGA GRVVLFSNPN TTSGRSHMTL KASTNDAGSW PEDKWLLYDA RKGWGYSCLA
              650 660 670
PVDKNHVGVL YESQGALNFL KIPYKDVLNA KNAR
SEQ ID NO: 25
                10 20 30 40 50 60 70 80
MKRNHYLFTL ILLLGCSIFV KASDTVFVHQ TQIPILIERQ DNVLFYFRLD AKESRMMDEI VLDFGKSVNL SDVQAVKLYY
                90 100 110 120 130 140 150 160
GGTEALQDKG KKRFAPVDYI SSHRPGNTLA AIPSYSIKCA EALQPSAKVV LKSHYKLFPG INFFWISLQM KPETSLFTKI
              170 180 190 200 210 220 230 240
SSELQSVKID GKEAICEERS PKDIIHRMAV GVRHAGDDGS ASFRIPGLVT SNKGTLLGVY DVRYNSSVDL QEYVDVGLSR
              250 260 270 280 290 300 310 320
STDGGKTWEK MRLPLSFGEY DGLPAAQNGV GDPSILVDTQ TNTIWVVAAW THGMGNQRAW WSSHPGMDLY QTAQLVMAKS
              330 340 350 360 370 380 390 400
TDDGKTWSKP INITEQVKDP SWYFLLQGPG RGITMSDGTL VFPTQFIDST RVPNAGIMYS KDRGKTWKMH NMARTNTTEA
              410 420 430 440 450 460 470 480
QVVETEPGVL MLNMRDNRGG SRAVIATKDL GKTWTEHPSS RKALQEPVCM ASLIHVEAED NVLDKDILLF SNPNTTRGRN
              490 500 510 520 530 540
HITIKASLDD GLTWPLEHQL MLDEGEGWGY SCLTMIDRET IGILYESSAA HMTFQAVKLK DLIR
```

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A recombinant oncolytic virus comprising a nucleotide sequence encoding a polypeptide comprising a sialidase domain, wherein the nucleotide sequence encoding the sialidase is operably linked to a promoter.

2. The oncolytic virus of claim 1, wherein said oncolytic virus is selected from the group consisting of: vaccinia virus, reovirus, Seneca Valley virus (SVV), vesicular stomatitis virus (VSV), Newcastle disease virus (NDV), herpes simplex virus (HSV), morbillivirus virus, retrovirus, influenza virus, Sinbis virus, poxvirus, measles virus, cytomegalovirus (CMV), lentivirus, and adenovirus.

3. The oncolytic virus of claim 2, wherein said oncolytic virus is a poxvirus, and said poxvirus is a vaccinia virus.

4. The oncolytic virus of claim 3, wherein the vaccinia virus is selected from among Dryvax; Lister; M63; LIVP; Tian Tan; Modified Vaccinia Ankara; New York City Board of Health, Dairen, Ikeda, LC16M8, Tashkent, IHD-J, Brighton, Dairen I, Connaught, Elstree, Wyeth, Copenhagen, and Western Reserve strains; vaccinia virus strain Elstree, vaccinia virus strain CL, vaccinia virus strain Lederle-Chorioallantoic, vaccinia virus strain AS.

5. The oncolytic virus of claim 1, wherein the sialidase is a human sialidase or a bacterial sialidase.

6. The oncolytic virus of claim 1, wherein the sialidase is a Neu5Ac alpha(2,6)-Gal sialidase or a Neu5Ac alpha(2,3)-Gal sialidase.

7. The oncolytic virus of claim 1, wherein the human or bacterial sialidase is selected from the group consisting of: Clostridium perfringens sialidase, Actinomyces viscosus sialidase, Arthrobacter ureafaciens sialidase, NEU2, and NEU4.

8. The recombinant oncolytic virus of claim 1, wherein the promotor is a viral early promoter.

9. The recombinant oncolytic of claim 2, wherein the oncolytic virus is a poxvirus and the promoter is a poxvirus early promoter.

10. The recombinant oncolytic virus of claim 1, wherein the oncolytic virus is Talimogene Laherparepvec.

11. The recombinant oncolytic virus of claim 1 wherein the virus is a reovirus.

12. The recombinant oncolytic virus of claim 1, wherein the virus is an adenovirus having an E1ACR2 deletion.

13. The recombinant oncolytic virus of claim 1, wherein the sialidase is DAS181.

14. The recombinant oncolytic virus of claim 1, wherein the sialidase comprises the amino acid sequence of SEQ ID NO:1 or SEQ ID NO:2.

15. The recombinant oncolytic virus of claim 1 wherein the virus is vaccinia virus Western Reserve.

16. A method of treating solid tumor comprising administering to a patient in need thereof the recombinant virus of claim 1.
